(19) United States
(12) Patent Application Publication
(10) Pub. No.: US 2023/0218919 A1
(43) Pub. Date: Jul. 13, 2023

(54) THERAPEUTIC LIGHTED CATHETERS

(71) Applicant: The Research Foundation for the State University of New York, Albany, NY (US)

(72) Inventors: Dmitriy Nikolavsky, Jamesville, NY (US); Gennady Bratslavsky, Fayetteville, NY (US)

(21) Appl. No.: 18/095,121

(22) Filed: Jan. 10, 2023

Related U.S. Application Data
(60) Provisional application No. 63/297,988, filed on Jan. 10, 2022.

(51) Int. Cl. A61N 5/06

(52) U.S. Cl. CPC A61N 5/0601; A61N 2005/065; A61N 2005/0658

(57) ABSTRACT

A therapeutic lighted catheter that can provide therapeutic light within a biological fluid passage for purposes of treatment and/or curing of materials within the passage. The catheter includes an elongated shaft having a first end and a second end positioned opposite the first end, the elongated shaft including a lumen extending substantially therebetween, and at least one light emitting element positioned within the elongated shaft. A control system is in electronic communication with the light emitting element and provides power thereto such that the powered light emitting element applies a light within the biological fluid passage for therapy and/or curing. The catheter can also include a means to apply a curable material.

### CROSS-REFERENCE TO RELATED APPLICATIONS

This application claims the benefit of U.S. Provisional Patent Application No. 63/297,988, filed on Jan. 10, 2022, there entirety of which is hereby incorporated herein by this reference.

### BACKGROUND OF THE INVENTION

### 1. Field of the Invention

Embodiments of the present disclosure generally relate to the field of catheters more particularly, to catheters embedded with light therapy technology as to treat and prevent the development of scarring, narrowing, and/or strictures in hollow tubular structures.

### 2. Description of the Related Art

Catheters are commonly used in emergency room and clinics to drain and/or remove material (e.g., fluids) from areas within the body. In particular, catheters are very useful in urology and can be placed within the urethra and bladder for medical treatment. Catheters are used when there is a narrowing in urinary tract, urethra or ureter or the bladder neck. These narrowing's can result due to a urethral or ureteral stricture, bladder neck stenosis, anastomotic contractures, scar tissue, and other problematic conditions.

Usually, a urologist will diagnose the issue and determine a treatment. The most common treatment for urethral stricture is a urethral dilation, or a mechanical stretching of the location of the stricture or narrowing, followed by the placement of a catheter for a designated amount of time. While these treatments are considered minimally invasive, their success rate of about ten percent, as the treatment does not treat the underlying condition. Further, the current procedure involves using mechanical processes to widen the constricted areas, which by itself can result in further scaring leading to narrowing in the future.

Although there are other innovative treatments including urethroplasties, the treatments are significantly more invasive, require special surgical training, and require the catheter to remain in place for a longer period of time. As such a device is needed which provides both treatment of existing strictures, and other contractures in other hollow tube structures (e.g. Ureter, trachea, esophagus, etc.), while providing prevention of future recurrences of the injury.

### SUMMARY OF THE INVENTION

Briefly described, the invention is a therapeutic lighted catheter that can provide therapeutic light within a biological fluid passage for purposes of treatment and/or curing of materials within the passage. The catheter includes an elongated shaft having a first end and a second end positioned opposite the first end, the elongated shaft including a lumen extending substantially therebetween, and at least one light emitting element positioned within the elongated shaft. A control system is in electronic communication with the light emitting element and provides power thereto such that the powered light emitting element applies a light within the biological fluid passage for therapy and/or curing. The catheter can also include a means to apply a curable material.

In some embodiments the elongated shaft may also include a balloon inflation shaft integral with the elongated shaft. The balloon inflation shaft may protrude into the fluid drainage lumen, while in other embodiments the balloon inflation shaft may protrude from the outer surface of the elongated shaft. In some embodiments the balloon inflation shaft may be in fluid communication with a balloon located about the second end of the elongated shaft. The balloon inflation shaft may also be in fluid communication with a balloon inflation port formed adjacent the first end of the elongated shaft.

In some embodiments the elongated shaft of the catheter may be constructed from a polymer. In some embodiments the polymer may be a soft plastic such as silicone rubber, latex or similar. In other embodiments nitinol, nylon, polyurethane, thermoplastic elastomers, and polyethylene terephthalate may be used. In some embodiments the elongated shaft may be formed from a plastic material, while in others it is formed from a synthetic material.

In some embodiments the light emitting element may be disposed between the inner surface and the outer surface. In other embodiments the light emitting element may be disposed integral to the outer surface, while in other embodiments the light emitting element may be disposed integral with the inner surface. In some embodiments the light emitting elements may be disposed concentrically with the inner surface and the outer surface.

In some embodiments, the light emitting element may be adjacent to the outer surface, while in other embodiments the light emitting element may be adjacent to the inner surface such that the inner surface protrudes into the fluid drainage lumen. Further, the light emitting element may be an internal rod extending through the elongated shaft. In one embodiment the rod may be a single light emitting element, while in others the light emitting element may be a plurality of light emitting elements concentrically surrounded by the elongated shaft.

In further embodiments, the light emitting element may be a plurality of light emitting elements. In some embodiments there may be more than one or several light emitting elements circumferentially spaced within the inner surface and outer surface. In some embodiments the light emitting element may be circumferentially wound between the inner surface and the outer surface. In some embodiments the light emitting elements may be directed to emit light in one direction.

In some embodiments, the light emitting element may be a light emitting diode. In other embodiments the light emitting element may be a laser. In some embodiments the light emitting element may be a series of light emitting diodes. In some embodiments, the light emitting element may emit an infrared light, while in others it may emit a red light, in further embodiments the light emitting diode may emit a near infrared light. In some embodiments, the light emitting element may emit a combination of a red light, a near infrared light and an infrared light, while in further embodiments the light emitting diode may emit red, near infrared, and infrared light.

In an embodiment, the light emitting element may span from the first end of the elongated shaft to the second end of the elongated shaft. In other embodiments the light emitting element may span a length between a first point on the elongated shaft and a second point on the elongated shaft, wherein the length between the first point and the second point is smaller than the elongated shaft.

In some embodiments the control system may be programed to provide individualized therapy to the patient based on patient characteristics or injury characteristics. In some embodiments the control system may provide power to the light emitting elements in a predetermined pattern, over a predetermined time frame.

In some embodiments the predetermined pattern may include flashing, pulses, strobing, or other lighting variations between each light emitting element. In some embodiment the therapy may be continuous over a span of one (1) to several days, while in other embodiments the therapy may be intermittent. The therapy may be applied once a day, twice a day, or another appropriate amount. In some embodiments the control system may be automatic, while in others the control system may require the user to turn it on and off.

In some embodiments the catheter may be a therapeutic lighted catheter for treatment and prevention of urethral strictures and bladder neck contractures, while in others it may be used for other hollow structures, e.g. Ureter, trachea, esophagus, etc.

In some embodiments, the fiber optics line may be formed adjacent to the fluid drainage lumen of the elongated shaft, while in other embodiments the fiber optics line may be formed integral with the elongated shaft. In further embodiments the elongated shaft may include a wall having an inner surface and an outer surface concentrically surrounding the inner shaft, where in the fiber optics line may protrude from the inner surface into the fluid drainage lumen. In further embodiments the fiber optics line may be circumferentially wound with the wall of the elongated shaft.

In some embodiments, the at least one fiber optics lines may be a plurality of fiber optics lines. The plurality of fiber optics lines may be formed adjacent to the one another, opposing each other, circumferentially placed, or other appropriate configurations.

In a further embodiment, the catheter may include a light source in electrical communication with the fiber optics line. In some embodiments the light source is may be light emitting diode, a fluorescent light, or other appropriate light. In some embodiments the light source may emit a red light, while in others the light source may emit an infrared light or near infrared light. In a further embodiment the light source may emit a combination of a red light, near infrared light, and an infrared light. In some embodiments the light source may toggle between emitting a red light, and an infrared light.

In some embodiments, the light source may be in electrical communication with a power source. The power source may be used turn the light source on and off, to apply the treatment as needed. In a further embodiment the light source may be in electrical communication with a control system. In this embodiment the power source may be integral with the control system. In some embodiments the control system may provide light to the fiber optics line at predetermined intervals. The predetermined intervals may be based on the patient characteristics, or the injury or treatment characteristics.

In some embodiments the catheter may further include a balloon inflation lumen integral with the fluid drainage lumen. The balloon inflation lumen may be in fluid communication with a balloon disposed around the second end of the elongated shaft. In some embodiments the balloon inflation lumen may be further removably fixed to a balloon inflation port.

In some embodiments, the light therapy may be applied at predetermined time intervals. The predetermined time interval may range from once an hour to once a day, more preferably once every few hours. The control system may apply the light therapy at predetermined light intensities. In some embodiments the control system may apply the light therapy in predetermined patterns. The predetermined pattern may include flashing, strobing, or pulsing.

In some embodiments, the control system may apply power to groups of the light emitting elements at different time intervals. In some embodiments the control system may provide power to a first group of the plurality of the light emitting elements at a first time and a second group of the plurality of light emitting elements at a second time, distinct from the first time to form a predetermined pattern of applied light therapy.

In a further embodiment, the invention includes a method of curing material within a biological fluid passage with a lighted catheter by inserting a catheter into a biological fluid passage, the catheter including an elongated shaft having a first end and a second end positioned opposite the first end, the elongated shaft including a lumen extending substantially between the first end and the second end therein, applying a curable material within the biological passage, and then curing the curable material with material-curative light emitted from at least one light emitting element positioned within the elongated shaft of the catheter. The method continues by controlling the curing with a control system in electronic communication with the light emitting element, where the control system is configured to provide power to the at least one light emitting element such that the powered light emitting element selectively emits material-curative light within a biological fluid passage.

The catheter can be embodied with a means of applying a curable material within a biological passage, and the method can include applying a curable material is applying the curable material through the means of the catheter. The curing of the curable material can be done by applying material-curative light at predetermined time intervals, and emitting at least one of red, near red, infrared, or ultraviolet light.

Other and further embodiments of the present disclosure are described below. The illustrative aspects of the present disclosure are designed to solve the problems herein described and/or other problems not discussed.

### BRIEF DESCRIPTION OF THE DRAWINGS

FIG. 1 is a perspective view of one embodiment of a catheter.

FIG. 2A is a front cross-sectional view of the catheter taken along line CS1-CS1 of FIG. 1.

FIG. 2B is a top cross-sectional view of the catheter taken along line CS2-CS2 of FIG. 1.

FIG. 3A is a front cross-sectional view of the catheter taken along line CS1-CS1 of FIG. 1.

FIG. 3B is a top cross-sectional view of the catheter taken along line CS2-CS2 of FIG. 1.

FIG. 4A is a front cross-sectional view of the catheter taken along line CS1-CS1 of FIG. 1.

FIG. 4B is a top cross-sectional view of the catheter taken along line CS2-CS2 of FIG. 1.

FIG. 5A is a front cross-sectional view of the catheter taken along line CS1-CS1 of FIG. 1.

FIG. 5B is a top cross-sectional view of the catheter taken along line CS2-CS2 of FIG. 1.

FIG. 6A is a front cross-sectional view of the catheter taken along line CS1-CS1 of FIG. 1.

FIG. 6B is a top cross-sectional view of the catheter taken along line CS2-CS2 of FIG. 1.

FIG. 7A is a front cross-sectional view of the catheter taken along line CS1-CS1 of FIG. 1.

FIG. 7B is a top cross-sectional view of the catheter taken along line CS2-CS2 of FIG. 1.

FIG. 8 is a perspective view of an alternative embodiment of a catheter.

FIG. 9 is a perspective view of an alternative embodiment of a catheter.

FIG. 10A is a front cross-sectional view of the catheter taken along line CS3-CS3 of FIG. 9.

FIG. 10B is a top cross-sectional view of the catheter taken along line CS4-CS4 of FIG. 9.

FIG. 11A is a front cross-sectional view of the catheter taken along line CS3-CS3 of FIG. 9.

FIG. 11B is a top cross-sectional view of the catheter taken along line CS4-CS4 of FIG. 9.

FIG. 12A is a front cross-sectional view of the catheter taken along line CS3-CS3 of FIG. 9.

FIG. 12B is a top cross-sectional view of the catheter taken along line CS4-CS4 of FIG. 9.

FIG. 13A is a front cross-sectional view of the catheter taken along line CS3-CS3 of FIG. 9.

FIG. 13B is a top cross-sectional view of the catheter taken along line CS4-CS4 of FIG. 9.

FIG. 14 is a perspective view of an alternative embodiment of a catheter.

FIG. 15 is a perspective view of an alternative embodiment of a catheter.

FIG. 16 is a perspective view of an alternative embodiment of a catheter.

FIG. 17 is a perspective view of one embodiment of a catheter including a fiber optic line and a lens.

FIG. 18A shows a front cross-sectional view of the catheter taken along line 18A-18A of FIG. 17.

FIG. 18B shows a front cross-sectional view of the catheter taken along line 18B-18B of FIG. 17.

FIG. 19 shows a perspective view of one embodiment of a catheter including a fiber optic line and a plurality of lenses.

FIG. 20 shows a perspective view of one embodiment of a catheter including a plurality of fiber optic lines and a lens.

FIG. 21A shows a front cross-sectional view of the catheter taken along line 21A-21A of FIG. 20.

FIG. 21B shows a front cross-sectional view of the catheter taken along line 21B-21B of FIG. 20.

FIG. 22 shows a perspective view of one embodiment of a catheter including a plurality of fiber optic lines and a lens.

FIG. 23 shows a perspective view of one embodiment of a catheter including a plurality of fiber optic lines and a lens.

FIG. 24A shows a front cross-sectional view of the catheter taken along line 24A-24A of FIG. 23.

FIG. 24B shows a front cross-sectional view of the catheter taken along line 24B-24B of FIG. 23.

FIG. 24C shows a front cross-sectional view of the catheter taken along line 24C-24C of FIG. 23.

FIG. 25 shows a perspective view of one embodiment of a catheter including a plurality of fiber optic bundles and a lens.

FIG. 26 shows a front cross-sectional view of the catheter taken along line 26-26 of FIG. 25.

FIG. 27 shows a perspective view of one embodiment of a catheter including a plurality of fiber optic bundles and a lens.

FIG. 28 shows a perspective view of one embodiment of a catheter including a fiber optic line and a plurality of lens.

FIG. 29 shows a front cross-sectional view of the catheter taken along line 29-29 of FIG. 28.

FIG. 30 shows a perspective view of one embodiment of a catheter including a fiber optic line and a plurality of lens.

FIG. 31 shows a perspective view of one embodiment of a catheter including a fiber optic line and a plurality of lens.

FIG. 32A shows a front cross-sectional view of the catheter taken along line 32A-32A of FIG. 31.

FIG. 32B shows a front cross-sectional view of the catheter taken along line 32B-32B of FIG. 31.

FIG. 33 shows a perspective view of one embodiment of a catheter system including a catheter section and a fiber optic section.

FIG. 34 shows an exploded perspective view of the catheter system of FIG. 33.

FIG. 35 shows a perspective view of one embodiment of a therapeutic lighted catheter.

FIG. 36 shows a schematic view of a urinary system for a patient with a therapeutic lighted catheter in situ.

FIG. 37 shows a cross-sectional front view of patient's ureter and elongated shaft of ureteral stent 4000 taken along line 37 in FIG. 36.

FIG. 38 shows another embodiment of a therapeutic lighted catheter in situ in a urinary system for a patient and ureteral stent.

FIG. 39 shows another embodiment of the therapeutic lighted catheter in situ within a urinary system of a patient and a nephroureteral stent.

### DETAILED DESCRIPTION

Embodiments of the present disclosure provides catheters for treating and preventing the development of scarring, narrowing, and/or strictures in hollow tubular structures. As used herein, the terms “axial” and/or “axially” refer to the relative position/direction of objects along axis (A), which is substantially parallel with the long axis of the elongated shaft discussed herein. As further used herein, the terms “radial” and/or “radially” refer to the relative position/direction of objects along axis (R), which is substantially perpendicular with axis (A) and intersects axis (A) at only one location. Additionally, the terms “circumferential” and/or “circumferentially” refer to the relative position/direction (C) of objects or features along a circumference which surrounds axis (A) but does not intersect the axis (A) at any location.

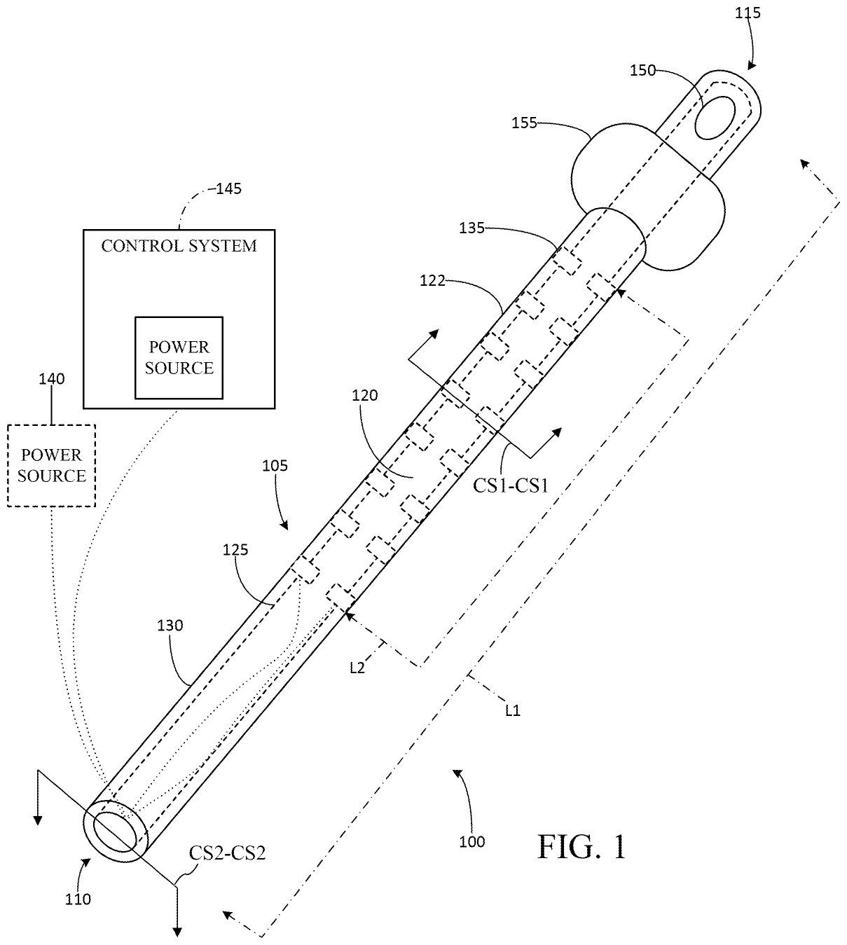

FIG. 1 shows a perspective view of a catheter 100 including an elongated shaft 105 having a first end 110 and a second end 115 formed, positioned, and/or disposed opposite first end 110. Elongated shaft 105 of catheter 100 may also include a fluid drainage lumen 120 extending substantially from first end 110 to second end 115. Fluid drainage lumen 120 may provide a channel in fluid communication with the internal, hollow tubular structure, (e.g., urinary tract, urethra, ureter, bladder, trachea, esophagus), wherein bodily fluids may flow from the hollow tubular structure, through fluid drainage lumen 120, and out into a collection device. Elongated shaft 105 may include and/or be formed as a wall 122. Wall 122 may include an inner surface 125 and an outer surface 130 circumferentially surrounding inner surface 125. As used herein, hollow tubular structure may refer to any body part, or other organic structure may that may suffer from scarring, narrowing, and/or strictures formed therein. Although various examples are given for the hollow tubular structure, it is understood that the listed structures are exemplary, and the hollow tubular structure may be any structure that may use or benefit from the use and/or treatment provided by catheter 100.

Catheter 100 may further include at least one light emitting element 135 positioned within elongated shaft 105. In some embodiments light emitting element 135 may be a plurality of light emitting elements axially spaced within wall 122 of elongated shaft 105. In other embodiments light emitting element 135 may be a single light emitting element. Light emitting element 135 may be a light emitting diode, a fluorescent light, or other acceptable form of light. Light emitting element 135 may emit light at a wavelength between 625 nm and 1 mm. More specifically light emitting element may emit an infrared light (780 nm-1 mm wavelength), near infrared light (750 nm-1400 nm wavelength), and/or a red light (625 nm-740 nm wavelength), however a light resulting from an appropriate wavelength suitable for treatment or prevention may be utilized.

Light emitting element 135 may be formed integral with elongated shaft 105. Light emitting element 135 may be adjacent to inner surface 125, or adjacent to outer surface 130. Light emitting element 135 may also be adjacent both inner surface 125, and outer surface 130. Further, light emitting element 135 may be formed within wall 122, between inner surface 125 and outer surface 130.

Elongated shaft 105 may include a first length (L1) extending from first end 110 to second end 115, and a second length (L2) extending at least partially between first end 110 and second end 115. As shown in FIG. 1, second length (L2) is shorter than first length (L1). Light emitting element 135 may be positioned within second length (L2) of elongated shaft 105.

Positioning of light emitting element 135 within second length (L2) and/or the size or length of second length (L2) may be based on characteristics of the patient receiving catheter 100. Some exemplary characteristics are gender, injury, stage of injury, or other prevalent characteristics.

FIG. 1 shows light emitting element 135 in electrical connection with a power source 140. In some embodiments power source 140 is a standalone device, while in other embodiments it may be integral with a control system 145. In some embodiments power source is a battery, an AC/DC current, or an electrical wire; however, any appropriate power source may be utilized. In a non-limiting example, power source 140 may be electrically coupled to each light emitting element 135 in series and/or light emitting element 135 may be arranged within elongated shaft 105 in series. In another non-limiting example, power source 140 may be electrically coupled in parallel and/or light emitting element 135 may be arranged in elongated shaft 105 in parallel. In additional non-limiting examples, power source 140 may be electrically coupled to each light emitting element 135 individually and may control the operation of each light emitting element 135 individually as discussed herein.

Control system 145 includes elements to control the luminosity of light emitting element 135, the intensity of light emitting element 135, the frequency of the treatment, the intervals when light emitting element 135 emits a light, and/or other operations of light emitting element 135.

Control system 145 may be a stand-alone system, or alternatively may be a portion and/or included in a larger computing device (not shown). As discussed herein, control system 145 may be configured to control light emitting elements 135 to aid in the operation of catheter 100 and/or aid in the treatment of the patient. As shown in FIG. 1, control system 145 may be in electronic communication with and/or communicatively coupled to various devices, apparatuses, and/or portions of catheter 100. In non-limiting examples, control system 145 be hard-wired and/or wirelessly connected to and/or in communication with power source 140, light emitting element 135, and its various components via any suitable electronic and/or mechanical communication component or technique. For example, control system 145 may be in electronic communication with light emitting element 135. Control system 145 may be in communication with power source 140 to control the power, intensity, intervals, and/or other operation of light emitting element 135, during the treatment process discussed herein. That is, and as discussed herein, once catheter 100 is deposited into internal hollow structure of the patient, control system 145 may instruct and/or operate power source 140 to apply power to light emitting element 135, which in turn may impart or apply a therapy to internal, hollow tubular structures. The therapy may be applied at predetermined time intervals. The time intervals may be from one (1) to several days, or any appropriate interval therein. In a non-limiting example catheter 100 may be a urinary tract catheter, wherein the light therapy may break up or widen strictures within the urethra. In a non-limiting embodiment, the urinary tract catheter may be used to treat injuries such as urethral stricture or bladder neck contracture. Additionally, and as discussed herein, control system 145 may also receive, process, and/or analyze inputs from various devices, portions, and/or sensors within catheter 100 to perform and/or optimize therapy via catheter 100.

Control system 145 may be programed based on individual characteristics of the patient and the treatment required therein. In some embodiments control system 145 may include a computer product in electrical communication with the light emitting element 135 such that control system provides therapy within a scheduled time frame. In some embodiments the control system 145 is an on/off switch wherein a user may selectively supply or not supply power to light emitting element 135. In some embodiments the control system provides power to light emitting element 135 at a predetermined intensity. In some embodiment the predetermined intensity may include a predetermined flashing pattern. In further non-limiting examples light emitting element 135 may provide a variety of flashing patters corresponding to the patient characteristics.

FIG. 1 further shows a catheter eye 150 in elongated shaft 105. Catheter eye 150 is in fluid communication with the internal, hollow tubular structure in which catheter 100 is inserted, and in fluid communication with fluid drainage lumen 120. First end 110 of elongated shaft may include a drainage port (not shown) in fluid communication with fluid drainage lumen 120. In some embodiments first end 110 is removably fixable to a fluid drainage port, wherein fluid drainage port is in fluid communication with fluid drainage lumen 120.

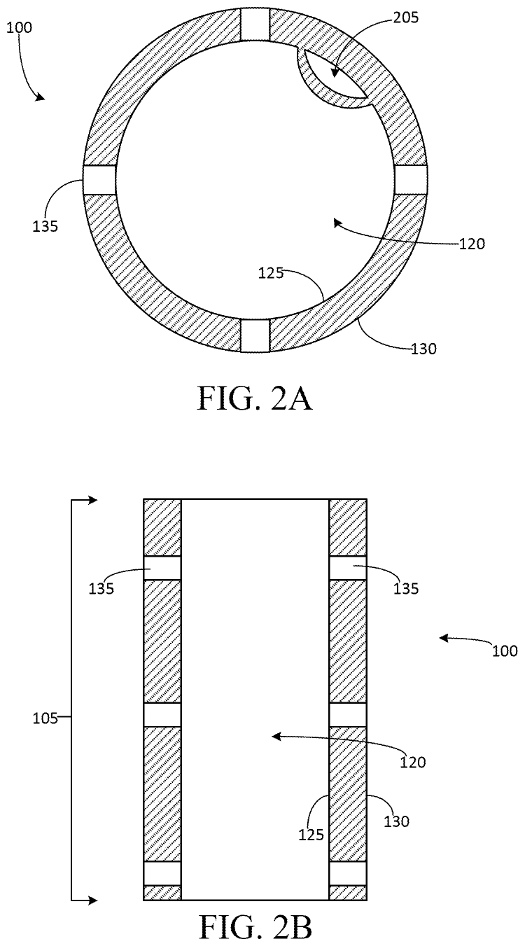

Catheter 100 may further include a balloon 155 disposed about second end 115 of elongated shaft 105. Balloon 155 is in fluid communication with a balloon inflation lumen, (see, FIG. 2A), which may be formed as a protrusion adjacent outer surface 130 of elongated shaft 105. balloon inflation lumen may also be formed as a protrusion adjacent inner surface 125 of elongated shaft 105.

Catheter 100 may be constructed from polymer. In some embodiments the polymer may be a soft plastic such as silicone rubber, latex, or a similar suitable material. In other embodiments nitinol, nylon, polyurethane, thermoplastic elastomers, and polyethylene terephthalate may be used. In some embodiments elongated shaft 105 may be formed from a plastic material, while in others it may be formed from a synthetic material.

FIG. 2A and FIG. 2B show an exemplary embodiment of catheter 100 shown in FIG. 1. More specifically, FIG. 2A shows a front cross-section view of an embodiment of catheter 100 taken from CS1-CS1, and FIG. 2B shows a top cross-section view of an embodiment of catheter 100. In a non-limiting example, there are a plurality of light emitting elements 135 circumferentially spaced adjacent to fluid drainage lumen 120. A non-limiting example shows four light emitting elements 135 disposed circumferentially within wall 122 at CS1-CS1. Light emitting elements 135 may be axially and/or radially aligned. Although, four light emitting elements 135 are shown any appropriate number may be used.

The embodiment shows a balloon inflation lumen 205 protruding from the inner surface 125 of wall 122. However, in other embodiments lumen 205 may be protruding from outer surface 130. Additionally, lumen 205 extends through lumen 120, in fluid communication with balloon 155. Lumen 205 may be removably fixed to a balloon inflation port, such that inflation port (not shown) is in fluid communication with balloon 155. Inflation port may provide air to balloon 155 via lumen 205 such that balloon 155 inflates to hold catheter 100 within the hollow tubular structure. In some embodiments, inflation port may be removably attached to lumen 205, while in others inflation port may be integral to lumen 205, or fixedly attached to lumen 205.

In a non-limiting example shown in FIG. 2B, light emitting elements 135 are formed or positioned parallel to each other within elongated shaft 105. In some embodiments light emitting elements 135 are evenly and axially spaced throughout elongated shaft 105, while in others light emitting elements 135 are concentrated in segments, e.g. a second length as described above of elongated shaft 105.

In a non-limiting example, light emitting elements 135 are formed integral to wall 122, wherein light emitting elements 135 are located between inner surface 125, and outer surface 130 of wall 122. In a non-limiting example light emitting elements 135 may be in a series within wall 122, wherein four series of light emitting elements 135 may be circumferentially spaced within wall 122 of elongated shaft 105. Although the present example uses four series of light emitting elements any appropriate number of light emitting elements may be used.

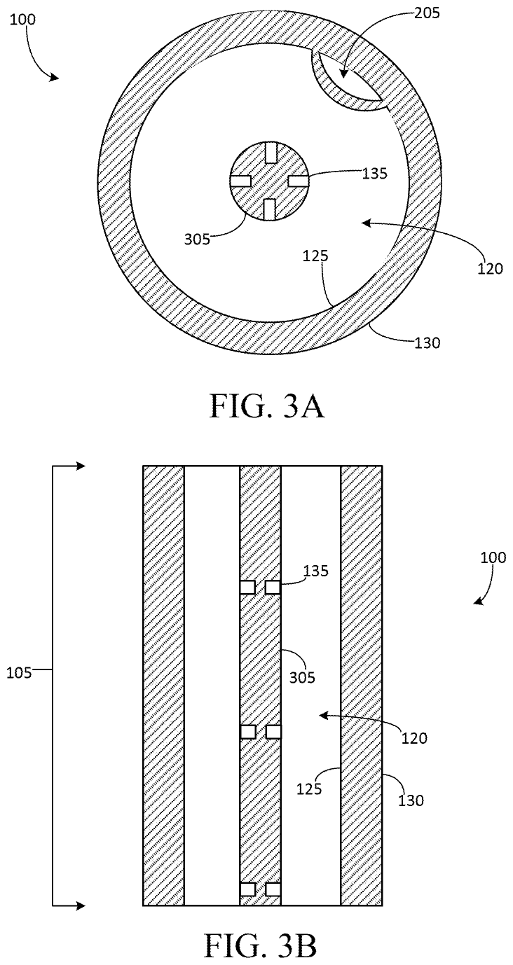

FIG. 3A and FIG. 3B show another non-limiting example of catheter 100. FIG. 3A shows a front cross-section view of an embodiment of catheter 100 taken from CS1-CS1, and FIG. 3B a top cross-section view of an embodiment of catheter 100. It is understood that similarly named components and/or similarly numbered components may function in a substantially similar fashion, may include similar materials/components, and/or may include similar interactions with other components. Redundant explanation of these components has been omitted for clarity.

In the non-limiting example, an internal rod 305 is positioned within the fluid drainage lumen 120. More specifically, internal rod 305 may be positioned/disposed within and/or may extend axially through fluid or air drainage lumen 120, adjacent inner surface 125 of wall 122. As such, wall 122 of elongated shaft 105 forming catheter 100 may substantially surround and/or be substantially concentric with internal rod 305. A plurality of light emitting elements 135 may be disposed within internal rod 305. In a non-limiting example, light emitting elements 135 may extend axially though internal rod 305. Although, FIG. 3A shows four light emitting elements 135 circumferentially spaced within internal rod 305 any appropriate number of elements may be used and/or may be radially aligned.

In some embodiments catheter 100 may have balloon inflation lumen 205 formed integral to inner surface 125 of wall 122, while in other embodiments balloon inflation lumen 205 may be formed integral to outer surface 130.

In a non-limiting example internal rod 305 can be made from a transparent material such that the light from light emitting elements 135 can go through internal rod 305 and catheter 100. Internal rod 305 may be removably fixed to elongated shaft 105 at first end 110, or second end 115. In some embodiments internal rod 305 may be inserted into catheter 100 through fluid drainage port, not shown, located adjacent to first end 110.

In the non-limiting example shown in FIG. 3B, light emitting elements 135 may be spaced axially from one another within internal rod 305. In further embodiments, light emitting elements 135 may be radially aligned within internal rod 305. In some embodiments light emitting elements 135 are disposed on a first length of internal rod 305, extending from first end 110 to second end 115. In other embodiments light emitting elements 135 are disposed on a second length of internal rod 305, wherein the second length extends partially between first end 110 and second end 115. The second length may be determined by characteristics of the patient. In some embodiments the characteristics can be one of gender, or patient specific injury.

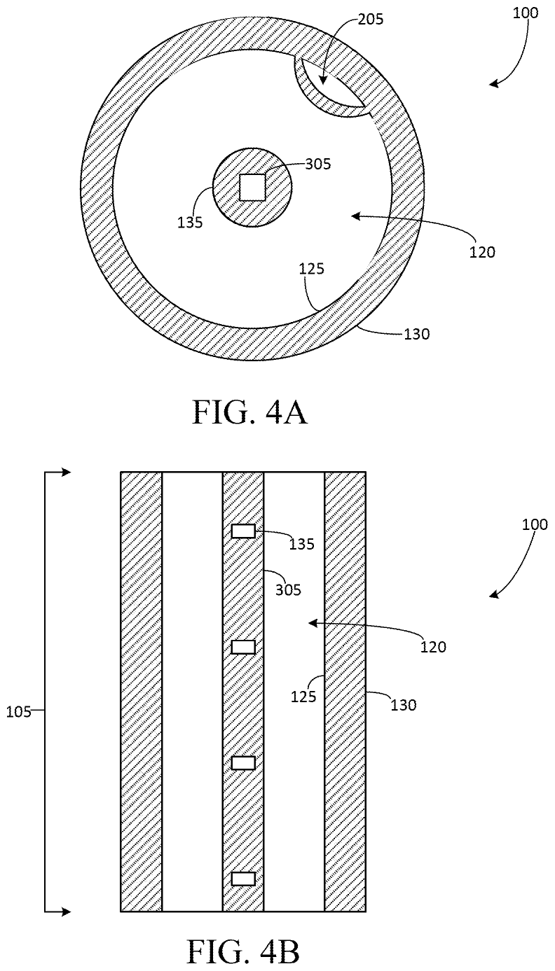

Catheter 100 shown in FIG. 4A and 4B may be substantially similar and/or include substantially similar features as those recited with respect to catheter 100 of FIGS. 3A and 3B. Redundant explanation of these features is omitted for brevity. Distinct from the non-limiting example shown and discussed herein with respect to FIGS. 3A and 3B, catheter 100 of FIGS. 4A and 4B may include a plurality of light emitting elements 135 disposed axially throughout internal rod 305. That is, a first light emitting element 135 may be positioned disposed, and/or formed integrally within internal rod 305 in a single or first axial position of internal rod 305 adjacent first end 110. Additionally, consecutive and/or subsequent light emitting elements 135 may be axially spaced from the first light emitting element 135, and may be positioned within and axially spaced throughout internal rod 305 from first end 110 to substantially adjacent second end 115.

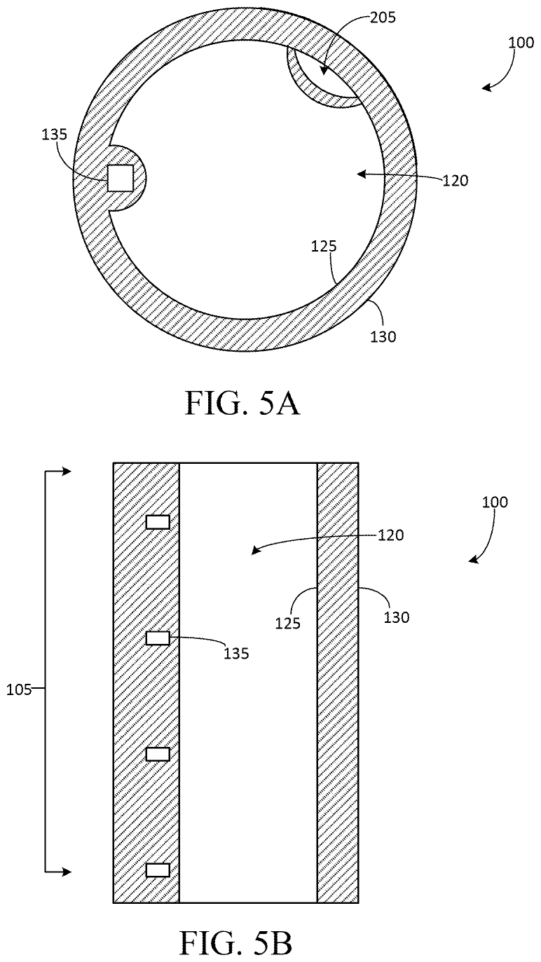

FIG. 5A and FIG. 5B show another exemplary embodiment of catheter 100. FIG. 5A shows a front cross-section view of an embodiment of catheter 100 taken from CS1-CS1. In a non-limiting example light emitting element 135 is adjacent to inner surface 125 of elongated shaft 105. More specifically, light emitting element may be formed integral in a protrusion that extends radially inward from inner surface 125 and/or into lumen 120. In a non-limiting example light emitting element 135 may be adjacent to, opposite of, or near balloon inflation lumen 205. In some embodiments inner surface 125 encases light emitting element 135 such that inner surface 125 protrudes into lumen 120.

FIG. 5B shows a top cross-section view of an embodiment of catheter 100 taken from CS2-CS2. In a non-limiting example as light emitting element 135 protrudes from a section of inner surface 125, there is a section of elongated shaft 105 wherein inner surface of wall 122 is thicker in an area surrounding light emitting element 135.

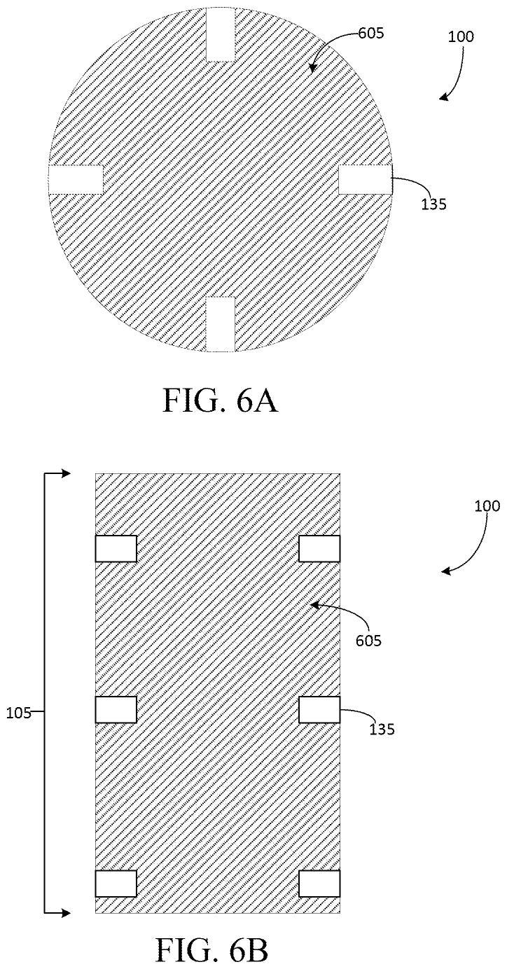

FIG. 6A and FIG. 6B show another exemplary embodiment of catheter 100. FIG. 6A shows a front cross-section view of an embodiment of catheter 100 taken from CS1-CS1 and FIG. 6B show a top cross-section view of an embodiment of catheter 100 taken from CS2-CS2. In a non-limiting example catheter 100, is a single rod 605 with a plurality of light emitting elements 135. Single rod 605 is a unitary body having no lumen therein. Light emitting elements 135 may be circumferentially spaced. More specifically, light emitting elements may be radially aligned. In the embodiment four light emitting elements 135 are shown, however any appropriate number of light emitting elements 135 may be used. Light emitting elements 135 are outward facing such that light is emitted outward of single rod 605.

Single rod 605 may be constructed from a transparent and/or light transmitting material, for example plastic or another synthetic polymer, or single rod 605 may be constructed from a transparent/translucent flexible material.

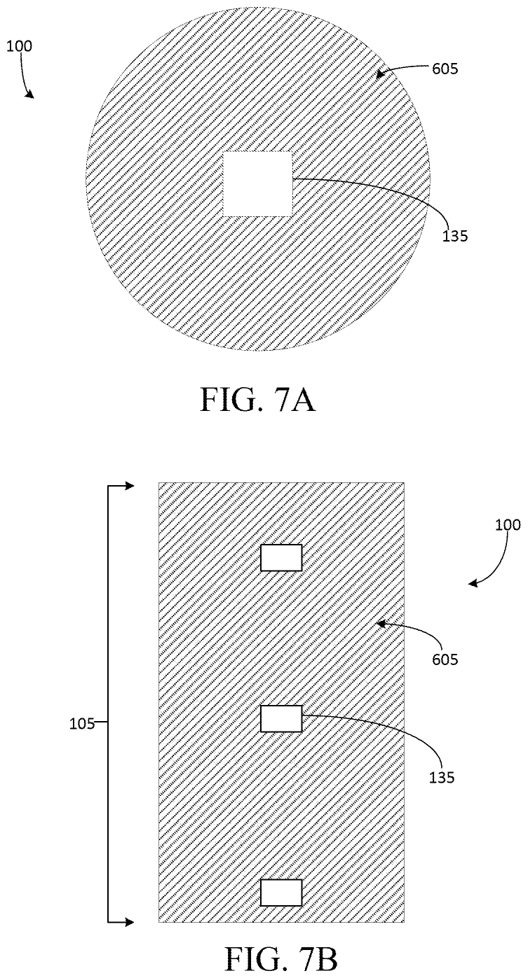

Turning to FIG. 6B, single rod 605 may have a plurality of light emitting elements 135 formed integral to single rod 605. Light emitting elements 135 may be axially spaced such that light emitting elements 135 extend substantially throughout single rod 605 from first end 110 to second end 115. In other embodiments light emitting element 135 may be integral to a second length of single rod 605, wherein second length extends partially between first end 110, and second end 115 of single rod 605. Second length may be determined by patient and/or treatment characteristics such as gender and/or injury. In a non-limiting example single rod 605 may be a light transmitting material, for example plastic or another appropriate synthetic polymer. Single rod 605 shown in FIG. 7A and 7B. may be substantially similar and/or include substantially similar features as those recited with respect to single rod 605 of FIGS. 6A and 6B. Redundant explanation of these features is omitted for brevity. Distinct from the non-limiting example shown and discussed herein with respect to FIGS. 6A and 6B, single rod 605 of FIGS. 7A and 7B may include a plurality of light emitting elements 135 disposed axially throughout single rod 605. That is, a first light emitting element 135 may be positioned disposed, and/or formed integrally within single rod 605 in a single or first axial position of single rod 605 adjacent first end 110. Additionally, consecutive, and/or subsequent light emitting elements 135 may be axially spaced from the first light emitting element 135 and may be positioned within and axially spaced throughout single rod 605 from first end 110 to substantially adjacent second end 115. Single rod 605 may surround light emitting element 135. As such single rod 605 may be made from a transparent/translucent material such that light emitted from light emitting element 135 may penetrate single rod 605 to reach internal, hollow tubular structure.

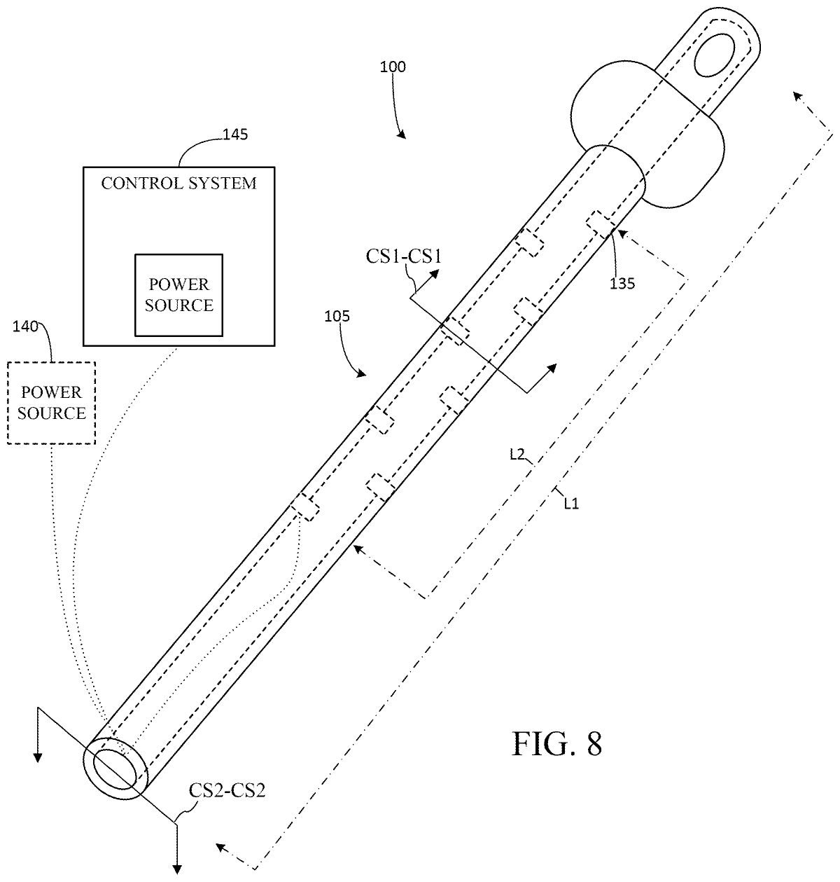

FIG. 8 shows an alternative embodiment of catheter 100. In a non-limiting example light emitting elements 135 may be staggered within elongated shaft 105. Light emitting elements 135 may be adjacent to inner surface 125, disposed within elongated shaft 105 (e.g., FIGS. 2A and 2B), disposed within internal rod 305 (e.g. FIGS. 3A 3B, 4A and 4B), or disposed on a single rod 605 (e.g. FIGS. 6A, 6B, 7A and 7B).

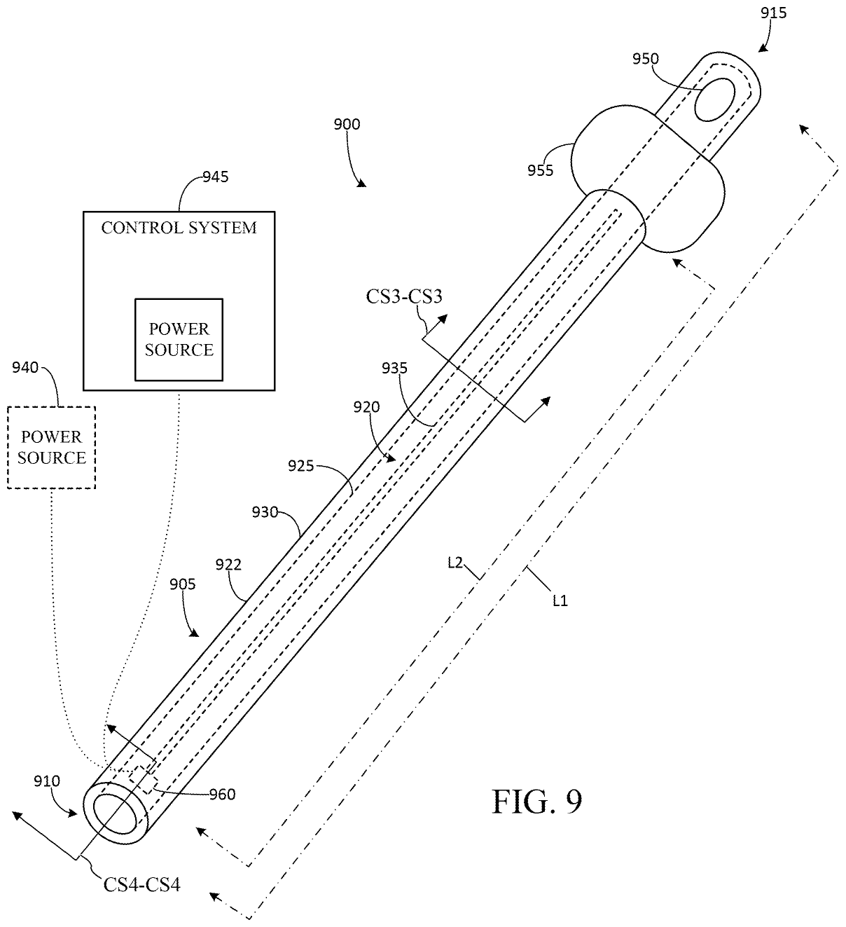

FIG. 9, shows an embodiment of catheter 900 that may include an elongated shaft 905 having a first end 910 and a second end 915 opposite the first end 910. Elongated shaft 905 of catheter 900 may also include a fluid drainage lumen 920 extending substantially from first end 910 to second end 915. Fluid drainage lumen 920 may provide a channel in fluid communication with the internal, hollow tubular structure, wherein bodily fluids or air may flow from internal hollow lumen structure through fluid/air drainage lumen 920 and out into a collection device (if needed). Elongated shaft 905 may include and/or be formed as a wall 922. Wall 922 may include an inner surface 925 and an outer surface 930 circumferentially surrounding inner surface 925.

The catheter 900 may further include at least one fiber optics line 935 positioned within elongated shaft 905. In some embodiments fiber optics line 935 is a plurality of fiber optics lines, while in other embodiments fiber optics line 935 is a single fiber optics line. Fiber optics line may include a light source 960 formed integral with elongated shaft 905. Light source 960 may be a light emitting diode, a fluorescent light, or other acceptable form of light. Light source 960 may emit light at a wavelength between 625 nm and 1 mm. More specifically light source 960 may emit an infrared light near infrared light, and/or a red light, however a light resulting from an appropriate wavelength suitable for treatment or prevention may be utilized.

Fiber optics line 935 may be formed integral with elongated shaft 905. Fiber optics line 935 may be adjacent inner surface 925, or adjacent outer surface 930. Fiber optics line 935 may also be adjacent both inner surface 925, and outer surface 930. Fiber optics line 935 may be formed integral to wall 922, between inner surface 925 and outer surface 930.

Positioning and/or the length of fiber optics line 935 may be based on characteristics of the patient receiving catheter 900. Some exemplary characteristics are gender, injury, stage of injury, or other prevalent characteristics.

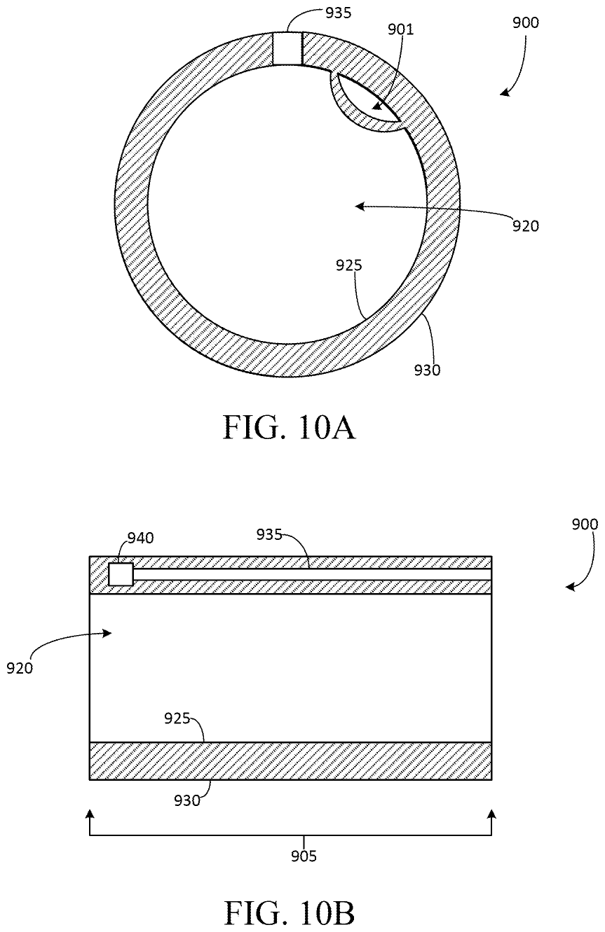

FIG. 9 shows fiber optics line 935 and/or light source 960 in electrical connection with a power source 940 and/or control system 945 similar to non-limiting examples discussed herein (e.g., FIG. 1). As discussed herein, light source 960 may provide and/or emit a light through fiber optics line 935. Also similarly discussed herein, control system 945 may control the operation of fiber optics line 935/light source 960 when providing light therapy to a hollow tubular structure of a patient. FIG. 9 may further include similar features and configurations to catheter 100, including a catheter eye 950, a balloon 955, and a balloon inflation lumen (see FIG. 10A).

FIG. 10A and FIG. 10B show an exemplary embodiment of catheter 900 show in FIG. 9. FIG. 10A shows a front cross-section view of an embodiment of catheter 900 taken from CS3-CS3. Catheter 900 includes a balloon inflation lumen 901, protruding from inner surface 925 of elongated shaft 905. A single fiber optics line 935 is shown integral to wall 922 of elongated shaft 905 between inner surface 925 and outer surface 930. In a non-limiting example fiber optics line 935 may be adjacent to balloon inflation lumen 901, however any appropriate placement including opposite of, near, or another location within elongated shaft 905 may be used.

FIG. 10B shows a top cross-section view of an embodiment of catheter 900 taken from CS4-CS4. Power source 940 may be within elongated shaft 905 in electrical connection with fiber optics line 935. Light source 960 may be integral with elongated shaft 905, and in electrical communication with power source 940.

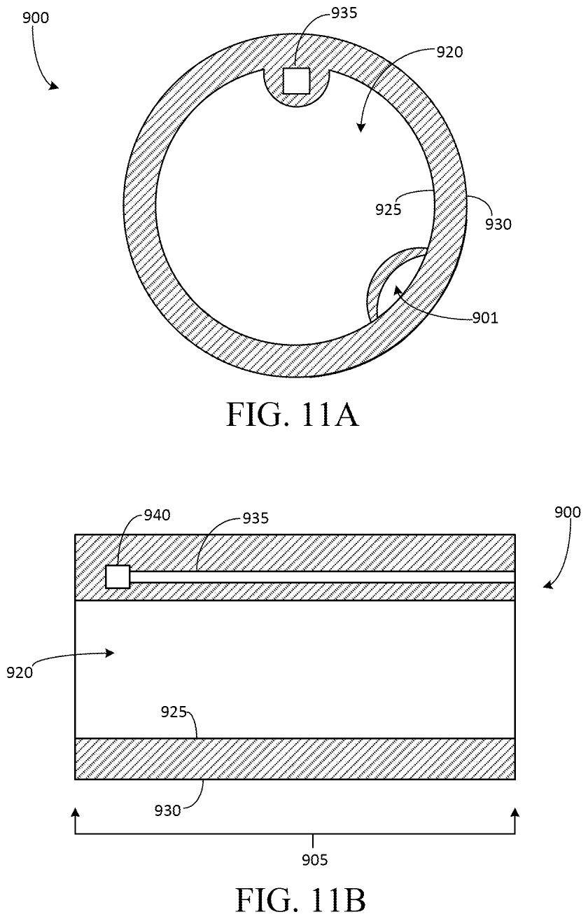

FIG. 11A and FIG. 11B show an exemplary embodiment of catheter 900 shown in FIG. 9. FIG. 11A shows a front cross-section view of an embodiment of catheter 900 taken from CS3-CS3. In a non-limiting example fiber optics line 935 may be formed integral in a protrusion that extends radially inward from inner surface 925 and/or into lumen 920. In a non-limiting example fiber optics line 935 may be adjacent to, opposite if, or near balloon inflation lumen 901. In some embodiments inner surface 925 encases fiber optics line 935 such that inner surface 925 protrudes into lumen 920.

FIG. 11B shows a top cross-section view of an embodiment of catheter 900 taken from CS4-CS4. In a non-limiting example as fiber optics line 935 protrudes from a section of inner surface 925, there is a section of elongated shaft 905 wherein wall 922 is thicker in an area surrounding fiber optics line 935.

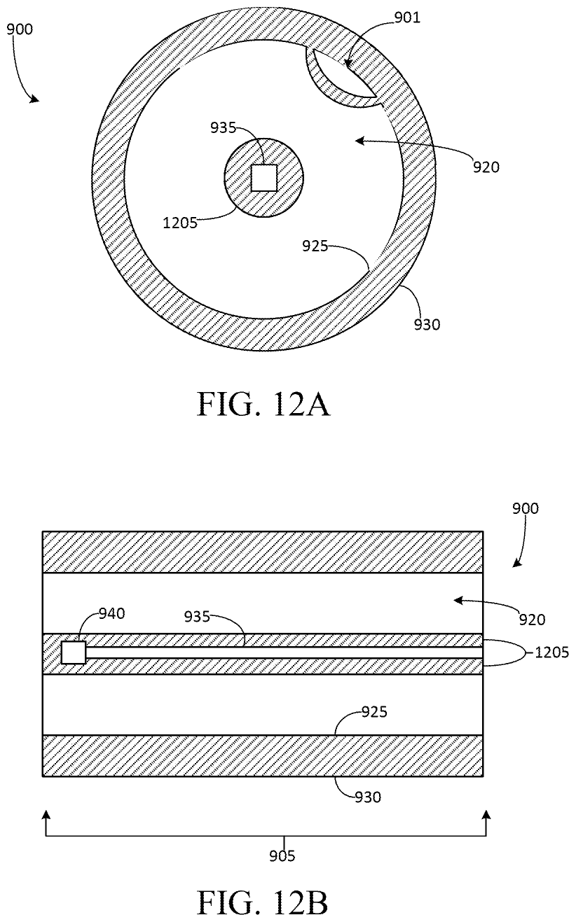

FIG. 12A and FIG. 12B show an exemplary embodiment of the catheter 900 shown in FIG. 9. FIG. 12A shows a front cross-section view of an embodiment of catheter 900 taken from CS3-CS3. Catheter 900 includes an internal rod 1205 positioned within fluid drainage lumen 920. More specifically, internal rod 1205 may be positioned/disposed within and/or may extend axially through fluid drainage lumen 920, adjacent inner surface 925 of wall 922. As such wall 922 of elongated shaft 905 forming catheter 900 may substantially surround and/or be substantially concentric with internal rod 1205. A fiber optics line 935 may be disposed within internal rod 1205. In a non-limiting example, fiber optics line 935 may extend axially through internal rod 1205. Internal rod 1205 may be made from a material which is transparent to light. In some embodiments, the material may be plastic, while in others the material may be a different synthetic polymer.

FIG. 12B shows a top cross-section view of an embodiment of catheter 900 taken from CS4-CS4. Power source 940 may be located within elongated shaft 905 in electrical communication with the light source 960. Internal rod 1205 may be removably fixed to elongated shaft 905 at first end 910, and/or second end 915. In some embodiments internal rod 1205 may be inserted to catheter 900 through fluid drainage port, not shown, located adjacent to first end 910.

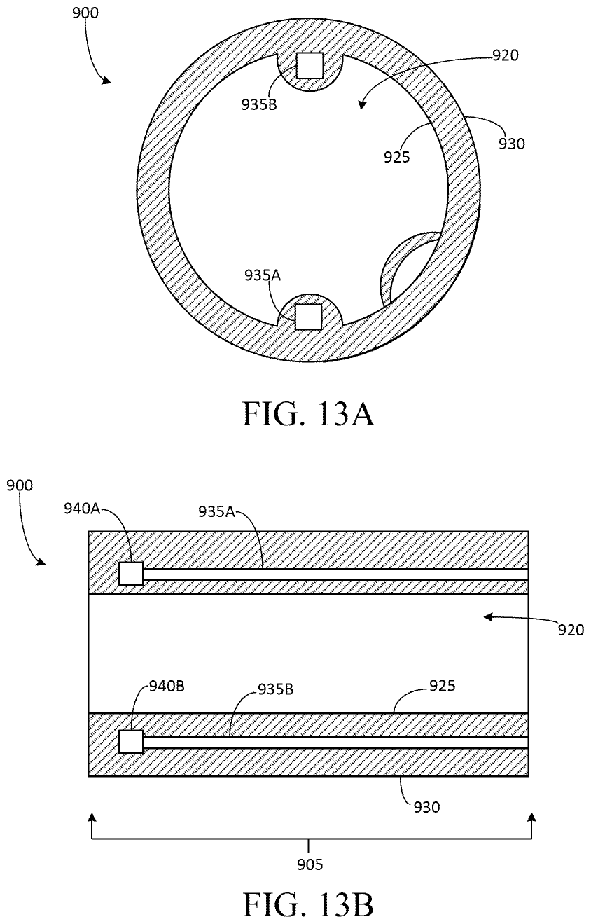

FIG. 13A and FIG. 13B show an exemplary embodiment of the catheter 900 shown in FIG. 9. FIG. 13A shows a front cross-section view of an embodiment of catheter 900 taken from CS3-CS3. The non-limiting embodiment includes two fiber optics lines 935A, 935B opposite each other. In some embodiments, fiber optics lines 935A, 935B may be adjacent or near each other for more targeted therapy. In some embodiments balloon inflation lumen 901 is adjacent to one of fiber optics lines 935A, 935B, however balloon inflation lumen 901 may be disposed along elongated shaft 905 between fiber optics lines 935A, 935B. In other embodiments balloon inflation lumen 901, and fiber optics lines 935A, 935B may be circumferentially placed about elongated shaft 905.

In a non-limiting example fiber optics lines 935A, 935B may be formed integral in a protrusion that extends radially inward from inner surface 925 and/or into lumen 920. In a non-limiting example fiber optics line 935A, 935B may be adjacent to, opposite if, or near balloon inflation lumen 901. In some embodiments inner surface 925 encases fiber optics line 935A, 935B such that inner surface 925 protrudes into lumen 920.

FIG. 13B shows a top cross-section view of an embodiment of catheter 900 taken from CS4-CS4. In a non-limiting example each fiber optics line 935A, 935B may be in electrical communication with an individual power source 940A, 940B. Each power source 940A, 940B may have a light source 960 integrally located within power source 940A, 940B.

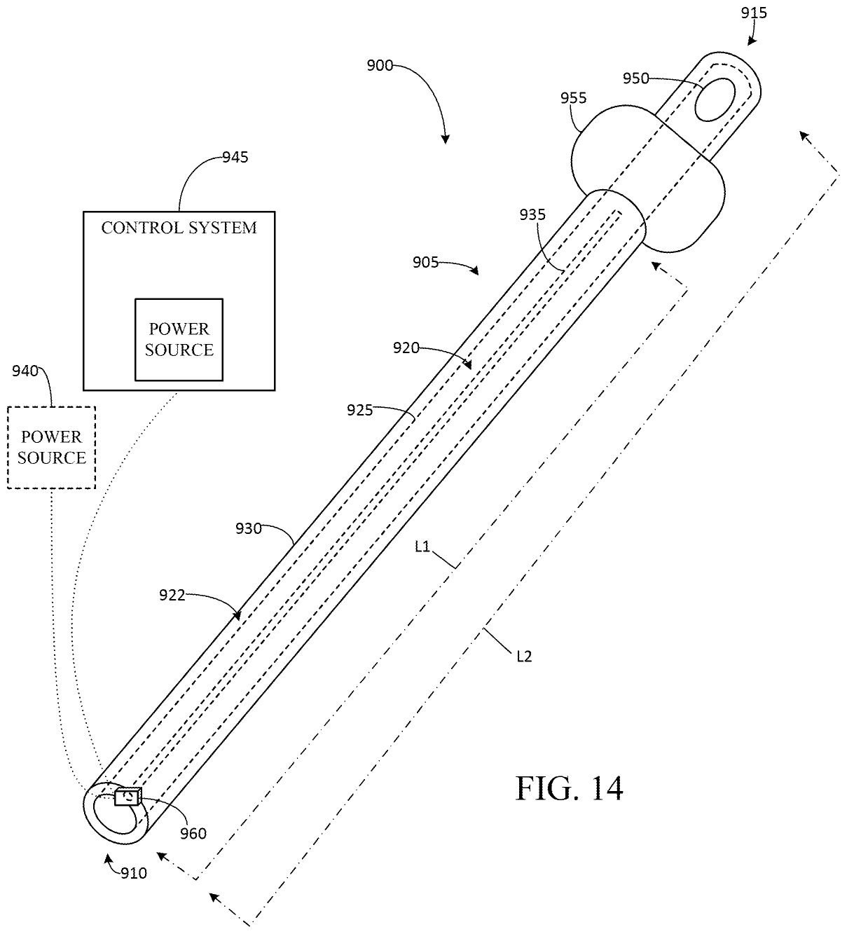

FIG. 14 is an exemplary embodiment of catheter 900. In a non-limiting example, light source 960 may be in communication with fiber optical line 935 outside of first end 910 of catheter 900. That is, and as shown in FIG. 14, light source 960 may not integrally formed or positioned within elongated shaft 905. Rather, light source 960 may be positioned outside of and/or formed distinct form elongated shaft 905, and thus may not be positioned within a patient's hollow tubular structure during operation of catheter 900, as discussed herein. Light source 960 may be in electrical communication with fiber optics line 935, and in electrical communication with power source 940. In some embodiments power source 940 is integral to control system 945, and light source 960 is in electrical communication with control system 945. Light source 960 may be a light emitting diode, a fluorescent light, or other appropriate light source. In some embodiments light source 960 may emit a light between 625 nm and 1 mm. More specifically light source 960 may emit a red light, near infrared light and/or an infrared light.

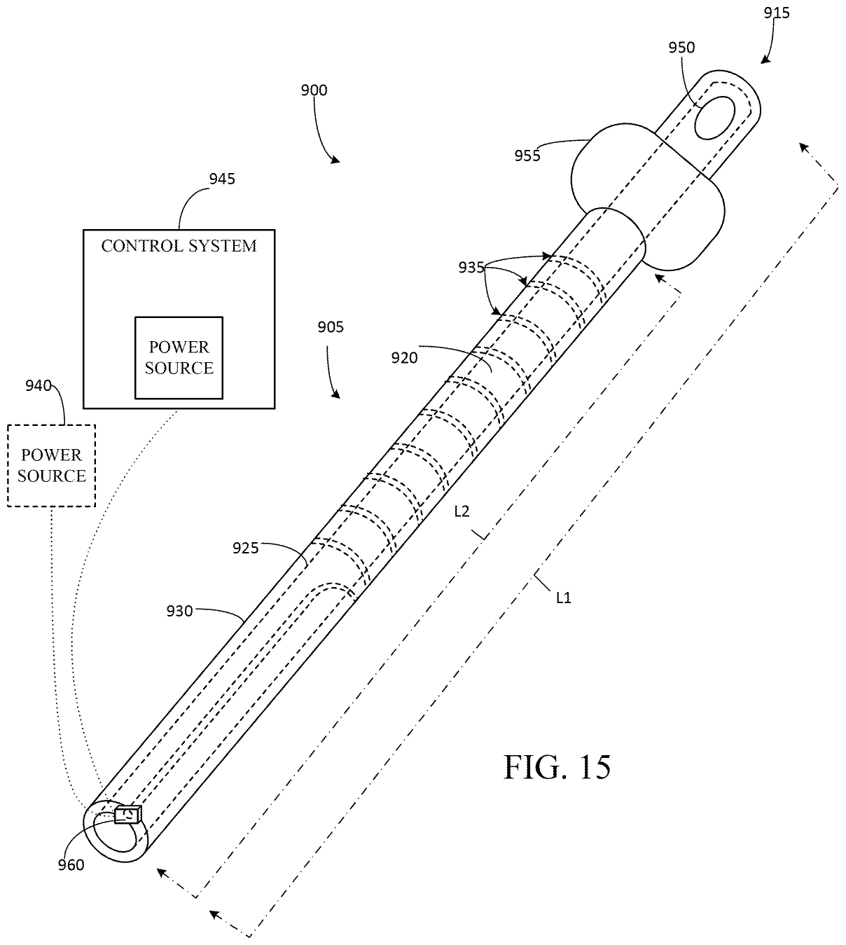

FIG. 15 is an exemplary embodiment of catheter 900. In a non-limiting example fiber optics line 935 is wound circumferentially within wall 922 between inner surface 925 and outer surface 930 of elongated shaft 905. The orientation of fiber optics line 935 may provide a uniform therapy around the circumference of catheter 900.

Further in a non-limiting example light source 960 is removably fixed to first end 910 of catheter 900. Light source 960 may be in electrical communication with fiber optics line 935, and in electrical communication with power source 940. In some embodiments power source 940 may be integrated into control system 945, and light source 960 may be in electrical communication with control system 945. Light source 960 may be a light emitting diode, a fluorescent light, or other appropriate light source. In some embodiments light source 960 may emit light at a wavelength between 625 nm and 1 mm. More specifically light source 960 may emit a red light, near infrared light and/or an infrared light.

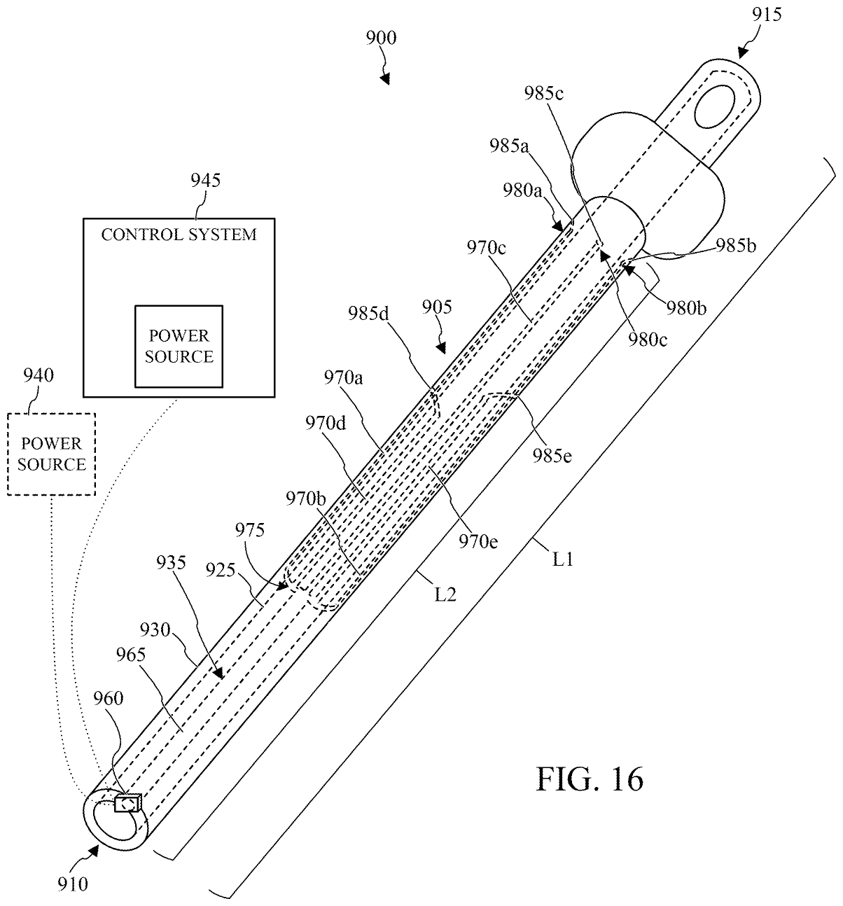

FIG. 16 is an exemplary embodiment of catheter 900. It is understood that similarly named components and/or similarly numbered components may function in a substantially similar fashion, may include similar materials/components, and/or may include similar interactions with other components. Redundant explanation of these components has been omitted for clarity.

In a non-limiting example, elongated shaft 905 may include a trunk 965 extending from first end 910 and at least partially through and/or within length L2. Trunk 965 may also extend through and/or be positioned within wall 922 of elongated shaft 905, adjacent drainage lumen 920. Trunk 965 may include a plurality of fiber optics lines 935 extending therein and/or therethrough. Trunk 965 may also split into a plurality of branches 970A, 970B, and 970C that may extend within a distinct portion of the second length L2 of elongated shaft 905. That is, elongated shaft 905 may also include a plurality of branches 970A, 970B, 970C that may extend from trunk 965 and/or may extend at least partially through elongated shaft 905 between first end 910 and second end 915. Similar to trunk 965, each of the plurality of branches 970A, 970B, 970C may extend through and/or be positioned within wall 922 of elongated shaft 905, adjacent drainage lumen 920. Each branch 970 may include and/or receive at least one distinct fiber optics line 935 extending through trunk 965. Each distinct branch 970 may have a proximal end 975 and a distal end 980, where in proximal end 975 is in physical and electrical communication with trunk 965. Red light, near infrared light, and/or infrared light may travel the length of branch 970 and shine out distal end 980 of branch 970. Distal end 980 may be about perpendicular to catheter 900, as to create an individual light point 985 on catheter 900 that may be positioned and/or formed directly adjacent outer surface 930 of wall 922. Resulting light points 985 on catheter 900 may emit a combination of red, near infrared, and infrared light, for treatment and prevention of injuries. In other non-limiting examples, trunk 965 may refer to a collection of fiber optics lines 935, and each of the plurality of branches 970 may refer to at least one distinct fiber optics line 935 included in trunk 965.

In some embodiments, branches 970 are circumferentially and radially spaced within wall 922, while in others branches 970 are randomly spaced within wall 922. In other non-limiting embodiments branches 970, may include further spits into micro branches, (not shown), wherein branches 970 and microbranches create a web like network of fiber optics lines 935. Although one trunk 965 and three branches 970A, 970B, 970C are shown, it is understood that catheter 900 may include more trunks 965 and/or more or less branches 970 extending from trunks 965.

In a non-limiting embodiment including multiple trunks 965, each trunk 965 (and/or the fiber optics lines 935 included therein) may be in electrical connection with a light source 960. Each light source 960 may emit light at a different wavelength, such that each trunk 965 and/or branches 970 extending therefrom may emit unique wavelengths. In a non-limiting example, a first light source 960A may emit a red light, while a second light source 960B may emit an infrared light, or vice versa. As such, first trunk 965A and corresponding branches 970 in catheter 900 electrically connected to first light source 960A may emit red light, while second trunk 965B and corresponding branches 970 in the same catheter 900 electrically connected to second light source 960B may emit infrared light. In some embodiments there may be a third trunk 965C and corresponding branches 970 in the same catheter 900 electrically connected to a third light source 960C may emit a near infrared light. Although three wave lengths are used in the foregoing example any corresponding number of wavelengths may be used.

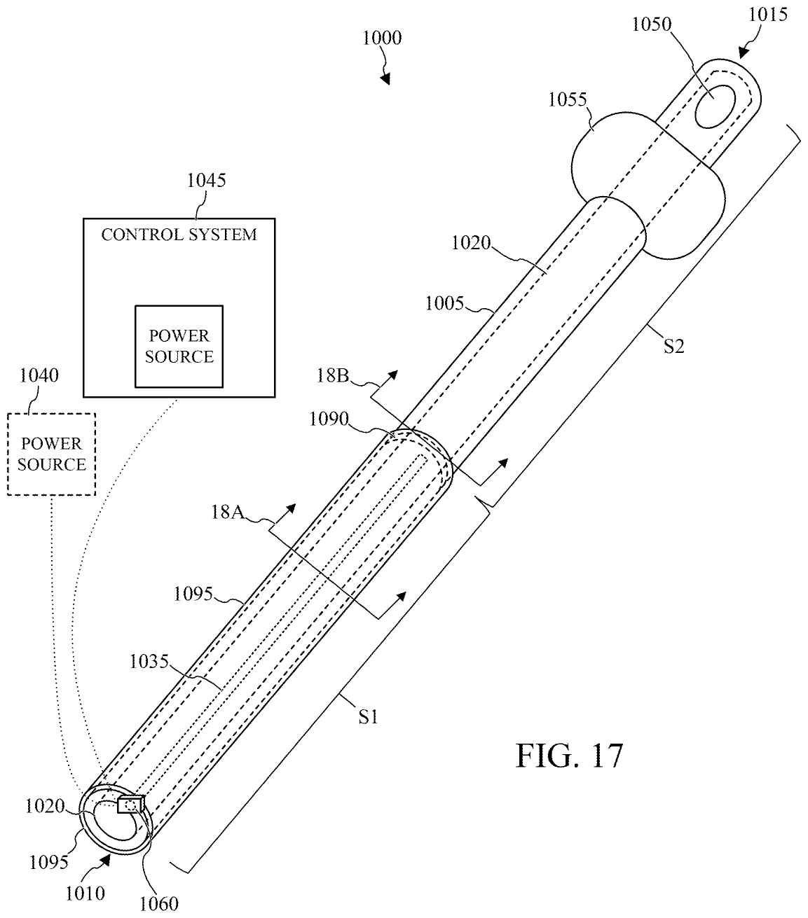

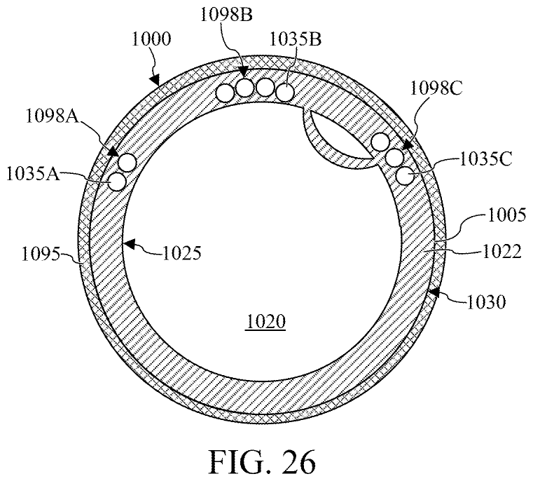

FIGS. 17-32B show various views of additional, non-limiting examples of catheter 1000 including at least one fiber optic line 1035. It is understood that similarly named components and/or similarly numbered components may function in a substantially similar fashion, may include similar materials/components, and/or may include similar interactions with other components. Redundant explanation of these components has been omitted for clarity.

FIG. 17 is a non-limiting embodiment of catheter 1000. In the non-limiting example, and similar to the embodiment shown in FIG. 14, at least one light source 1060 may be positioned outside of elongated shaft 1005 and adjacent to first end 1010 of elongated shaft 1005 of catheter 1000. Additionally, light source 1060 may be in electrical and/or optical communication with fiber optic line(s) 1035, and in electrical communication with power source 1040 and control system 1045. As discussed herein, light source 1060 may be a light emitting diode, a fluorescent light, or other appropriate light source. In some embodiments light source 1060 may emit a light between 625 nm and 1 mm. More specifically light source 1060 may emit a red light, near infrared light and/or an infrared light. As similarly discussed herein, control system 1045 may be in electronic communication with power source 1040 and light source 1060, and may be configured to control the operation of light source 1060. As discussed herein, control system 1045 may control the operation of light source 1060 by providing power to light source 1060, via power source 1040, to generate a light. The generated light may pass through fiber optic line 1035 and be disbursed through elongated shaft 1005 using at least one lens.

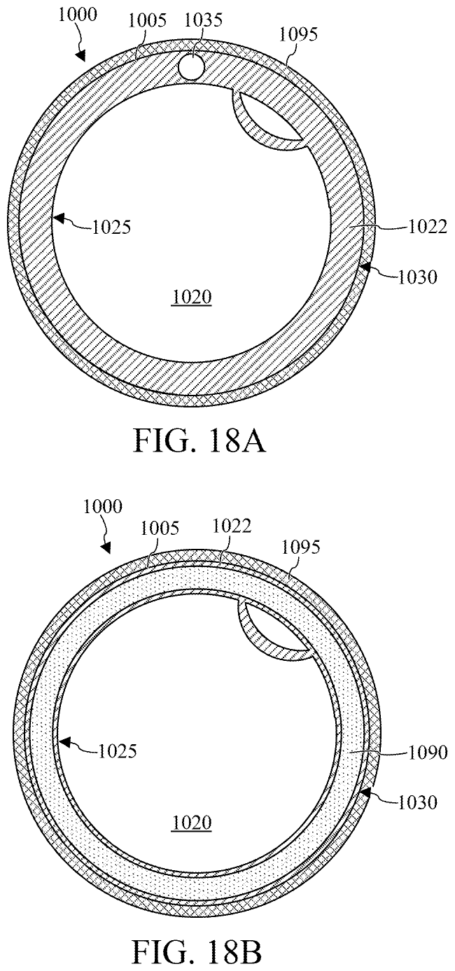

As discussed herein, at least one fiber optic line 1035 may be formed within elongated shaft 1005, adjacent fluid drainage lumen 1020. That is, and as shown in FIG. 18A, elongated shaft 1005 may include a wall 1022 that may substantially surround and/or define fluid drainage lumen 1020 extending axially through elongated shaft 1005. Wall 1022 may include inner surface 1025 and outer surface 1030. In the non-limiting example, fiber optic line 1035 may be disposed, formed, and/or included within wall 1022 of elongated shaft 1005. Additionally, fiber optic line 1035 may be formed, positioned, and/or disposed between inner surface 1025 and outer surface 1030 of elongated shaft 1005 as well. In the non-limiting example shown in FIG. 17, and as discussed herein, fiber optic line 1035 may extend only partially through elongated shaft 1005 from first end 1010, and may cease or end (directly) adjacent at least one lens included in catheter 1000.

As shown in FIGS. 17 and 18B catheter 1000 may also include at least one lens 1090. Lens 1090 may be positioned within elongated shaft 1005 between first end 1010 and second end 1015. More specifically, lens 1090 may be formed, positioned, disposed, and/or included in elongated shaft 1005 between first end 1010 and second end 1015, and may be disposed at least partially/circumferentially around and/or positioned adjacent to fluid drainage lumen 1020. Lens 1090 may be formed from any suitable optical lens or transmissive optical device that may disperse, spread, scatter, and/or diffuse the light generated by light source 1060 and provided to catheter 1000 via fiber optic line 1035. In the non-limiting example shown in FIGS. 17 and 18B, lens 1090 may be formed as a single, torus lens formed within elongated shaft 1005. As shown in FIG. 18B, torus lens forming lens 1090 may concentrically surround fluid drainage lumen 1020 of elongated shaft 1005. During operation, light source 1060 may provide light through fiber optic line 1035, which in turn may provide the generated light to lens 1090. Lens 1090 may then disperse, spread, scatter, and/or diffuse the light through the remainder of elongated shaft 1005, toward second end 1015 of catheter 1000. Because elongated shaft 1005 of catheter 1000 may be formed from a substantially clear, polymer material (e.g., silicone), the light dispersed from lens 1090 may shine, flow, and/or carry through elongated shaft 1005. In a non-limiting example, the length/depth/distance in which the generated light may penetrate elongated shaft 1005, as well as the light intensity/brightness within elongated shaft 1005 may be dependent upon, at least in part, the characteristics (e.g., intensity) of the light generated by light source 1060, characteristics of fiber optic line 1035 (e.g., length, size, number of fiber optic lines), and/or characteristics of lens 1090 (e.g., size, number, position). As such, the intensity of the light generated by light source 1060 to provide therapeutic relief to a stricture closest to the end of the urethra may be less than the intensity of the light generated to provide therapeutic relief to a stricture closest to the beginning of the urethra (e.g., adjacent the bladder).

Catheter 1000 may also include a cover 1095. As shown in FIG. 17, cover 1095 may be disposed over a portion of elongated shaft 1005. More specifically, cover 1095 may be disposed, enclosed, and/or substantially surround a first section (S1) of elongated shaft 1005. The first section (S1) of elongated shaft 1005 may include first end 1010 of elongated shaft 1005, lens 1090 formed within elongated shaft 1005, and fiber optic line 1035 formed within elongated shaft 1005. Cover 1095 may be formed from any suitable material that may block the generated light from escaping first section (S1) of elongated shaft 1005. Additionally, cover 1095 may also be formed from a material that may include reflective properties. As such, and during operation, any light that may pass from fiber optic line 1035 through first section (S1) of elongated shaft 1005 may be reflected back through elongated shaft 1005 and/or toward lens 1090.

As shown in FIG. 17, lens 1090 may be formed within elongated shaft 1005 at a predetermined distance away from second end 1015 of shaft 1005. As such, catheter 1000/elongated shaft 1005 may include a second section (S2) formed or positioned adjacent the first section (S1). Second section (S2) may not be covered by cover 1095, nor may it include lens 1090 or fiber optic lines 1035. Rather, second section (S2) may include the portion of catheter 1000 that may be inserted and positioned within a patient's urethra, while first section (S1), including cover 1095, lens 1090, fiber optic line 1035, and light source 1060 may remain outside of the patient's body. As such, second section (S2) may include a predetermined length based on characteristics of the patient. Characteristics of the patient may include, but are not limited to, gender, injury, stage of injury, or other prevalent characteristics.

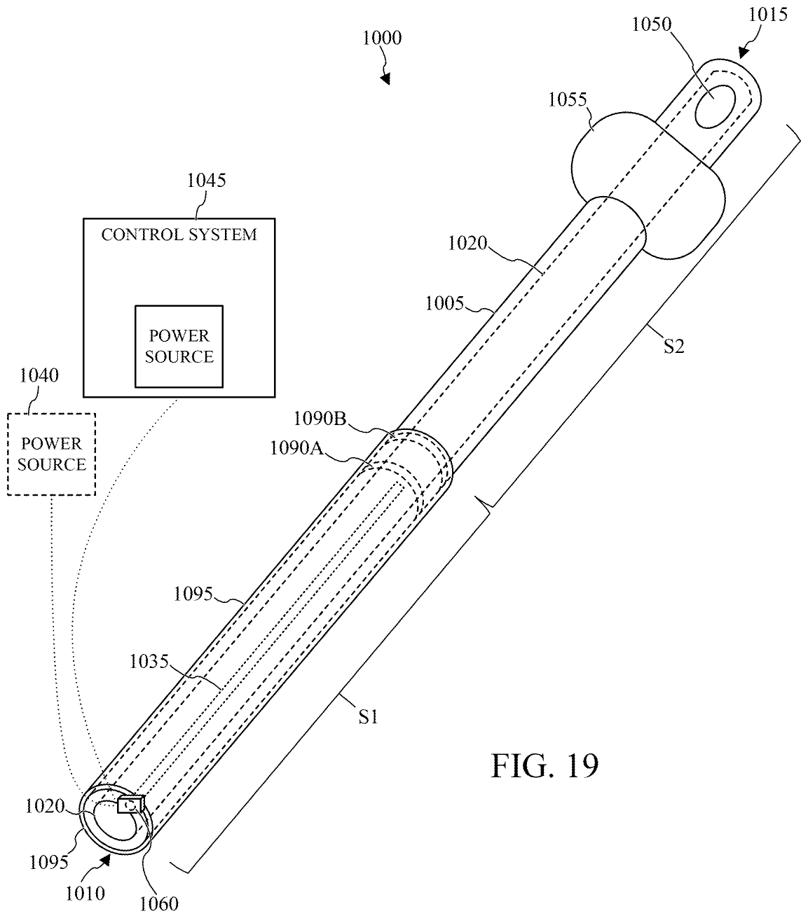

FIG. 19 shows another non-limiting example of catheter 1000. As shown, catheter 1000 may include a plurality of lenses 1090. In the non-limiting example shown in FIG. 19, catheter 1000 may a plurality of torus lenses 1090, where each torus lens may be positioned within first section (S1) of elongated shaft 1005. Additionally, each torus lens 1090 may be axially spaced apart form one another within elongated shaft 1005. That is, each of the plurality of lenses 1090 positioned within elongated shaft 1005 of catheter 1000 may be axially spaced or separated from one another to aid in the transmission, dissipation, and/or disbursement of light within second section (S2), as discussed herein. The (axial) spacing or distance between lenses 1090 within catheter may be predetermined and/or based on various characteristics including, but not limited to, characteristics (e.g., intensity) of the light generated by light source 1060, characteristics of fiber optic line 1035 (e.g., length, size, number of fiber optic lines), characteristics of lens 1090 (e.g., size, number, position), and/or characteristics of the patient.

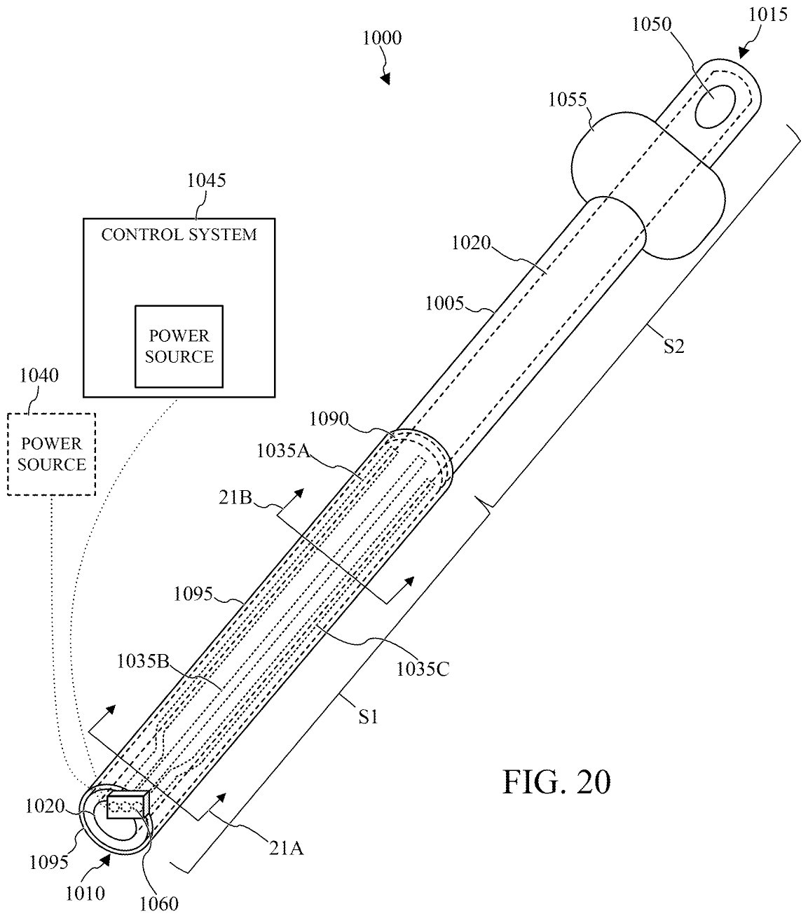

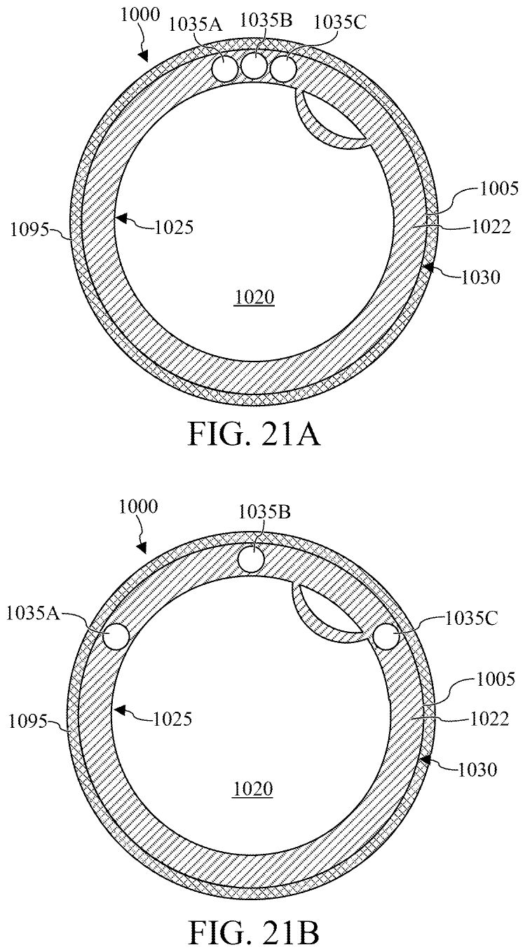

FIGS. 20-24C show various non-limiting examples of catheter 1000 including a plurality of fiber optic lines 1035. Turning to FIGS. 20-21B, elongated shaft 1005 of catheter 1000 may include a plurality of fiber optic lines 1035A, 1035B, 1035C. As shown in FIG. 20, each fiber optic line 1035A, 1035B, 1035C may extend axially within elongated shaft 1005 between first end 1010 and lens 1090. That is, each of the plurality of fiber optic lines 1035A, 1035B, 1035C may extend toward lens 1090 and/or may all be spaced a single, predetermined distance away from lens 1090 within second section (S2) of elongated shaft 1005. Additionally in the non-limiting example shown in FIGS. 21A and 21B, the plurality of fiber optic lines 1035A, 1035B, 1035C are disbursed within elongated shaft circumferentially around fluid drainage lumen 1020. As shown, catheter 1000 may include three distinct fiber optic lines 1035A, 1035B, 1035C. However, the number of fiber optic lines is illustrative. As such, it is understood that catheter 1000 may include more or less fiber optic lines than shown and discussed herein.

Additionally as shown in FIG. 20, catheter 1000 may include a single light source 1060. That is, a single light source 1060 may be in electrical/optical communication with each fiber optic line 1035A, 1035B, 1035C of the plurality of fiber optic lines. In the non-limiting example, each fiber optic line 1035A, 1035B, 1035C may converge near first end 1010 of elongated shaft 1005 in order to be in optical communication with and/or to receive generated light from light source 1060, as discussed herein. However, as each fiber optic line 1035A, 1035B, 1035C extends axially toward lens 1090, the circumferential spacing between fiber optic line 1035A, 1035B, 1035C may increase. The use of a plurality of fiber optic lines 1035A, 1035B, 1035C may increase the intensity of the therapeutic light delivered to the patient, as discussed herein.

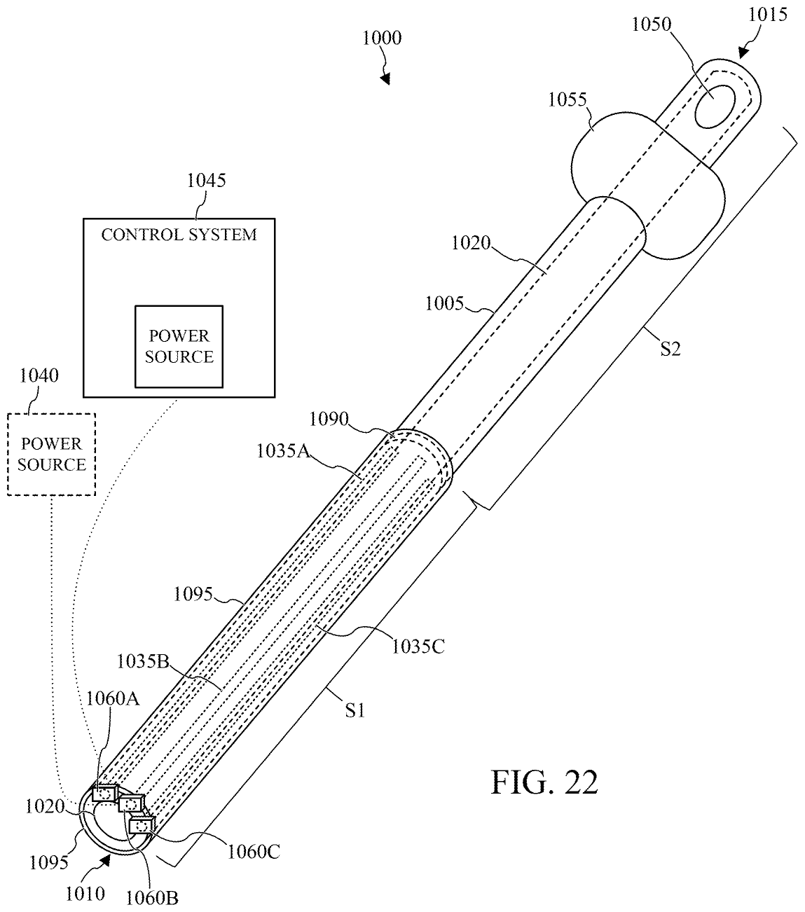

Distinct from the example shown in FIGS. 20-21B, catheter 1000 shown in FIG. 22 may include a plurality of light sources 1060. More specifically, catheter 1000 shown in FIG. 22 may include a plurality of light sources 1060A, 1060B, 1060C positioned outside of elongated shaft 1005 and adjacent to first end 1010 of elongated shaft 1005 of catheter 1000. Additionally, light sources 1060A, 1060B, 1060C may be in electrical and/or optical communication with a corresponding fiber optic line 1035A, 1035B, 1035C, and in electrical communication with power source 1040 and control system 1045. In the example shown, each light source 1060A, 1060B, 1060C may operate and/or be controlled independently of the other. As such, and dependent upon characteristics of the patient and/or lens 1090, the number and/or distinct light sources 1060A, 1060B, 1060C may generate light and subsequently provide the generated light to a corresponding fiber optic line 1035A, 1035B, 1035C. This may allow for improved control of the light therapy delivered to a patient by catheter 1000.

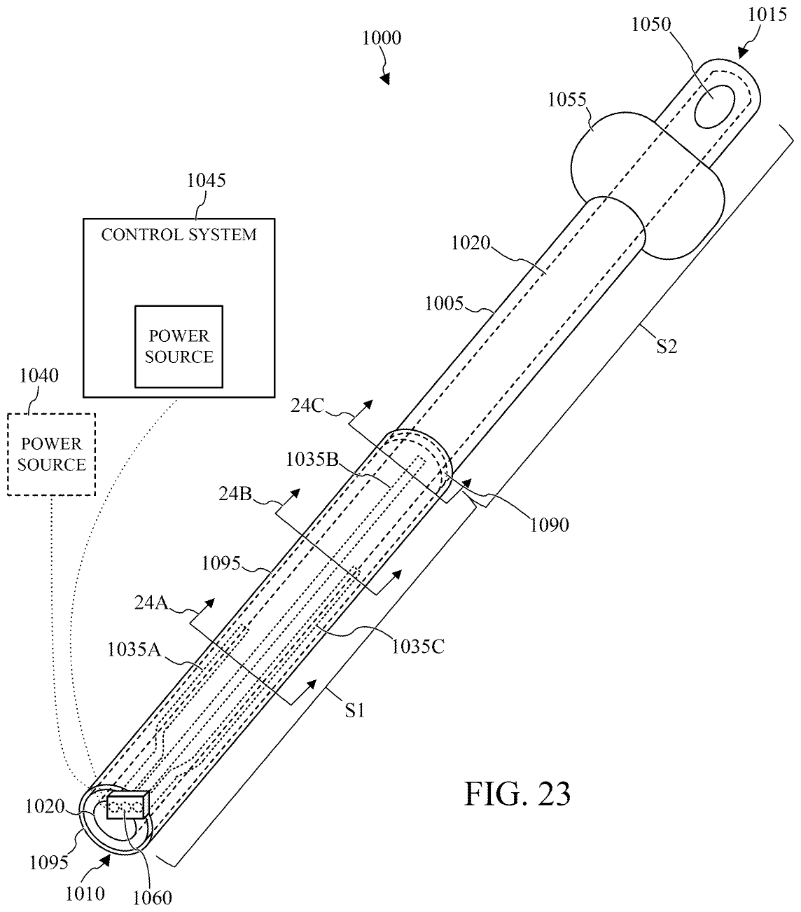

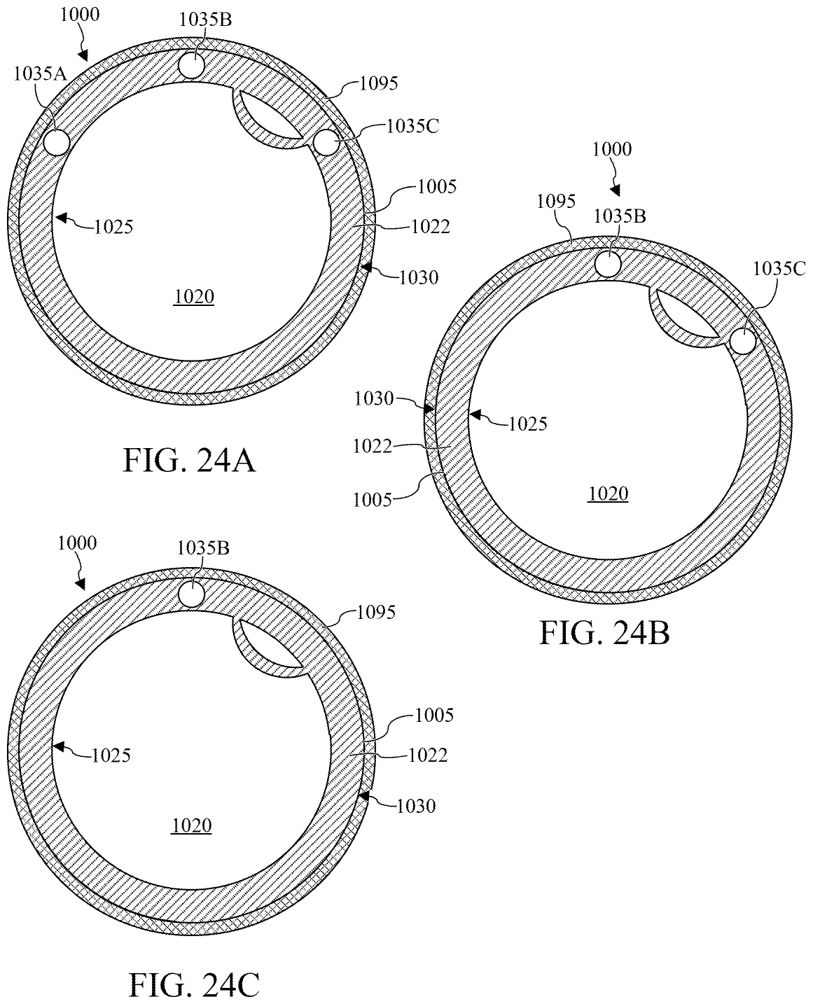

Turning to FIGS. 23-24C, each fiber optic line 1035A, 1035B, 1035C may extend axially within elongated shaft 1005 between first end 1010 and lens 1090. Distinct from the non-limiting example discussed herein with respect to FIG. 20, each of the plurality of fiber optic lines 1035A, 1035B, 1035C shown in FIGS. 23-24C may extend axially within elongated shaft 1005 from first end 1010 toward lens 1090 at a distinct distance. That is, each fiber optic line 1035A, 1035B, 1035C may include a distinct length and/or may end within elongated shaft 1005 at a distinct distance or length from lens 1090. For example, fiber optic line 1035B may extend to be directly adjacent lens 1090 (e.g., longest length), while the length of fiber optic line 1035A may be smaller than both fiber optic lines 1035B, 1035C.

Although shown as including a single light source 1060, it is understood that catheter 1000 shown in FIG. 23 may also include a plurality of light sources 1060A, 1060B, 1060C, as similarly discussed herein with respect to FIG. 22.

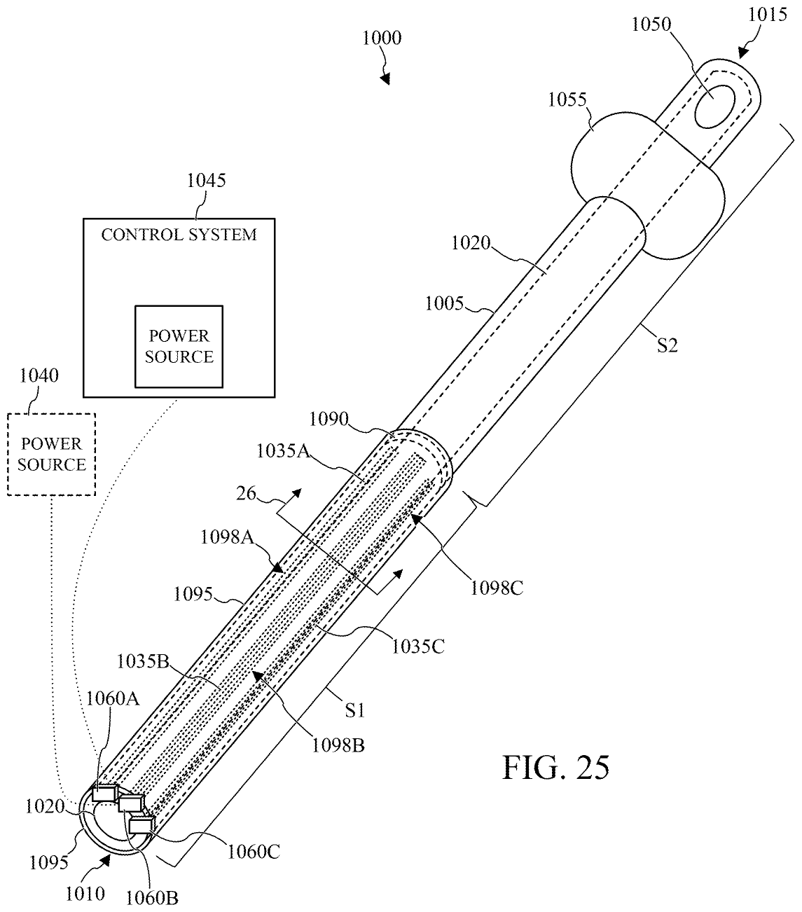

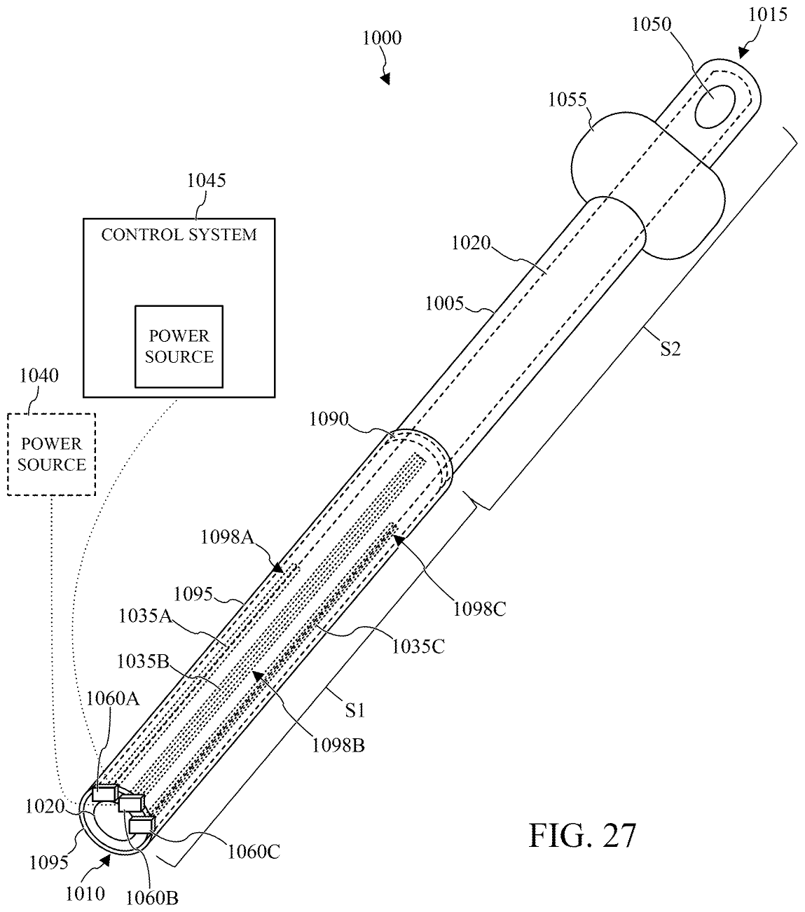

FIGS. 25-27 show various non-limiting examples of catheter 1000 including a plurality of fiber optic bundles 1098. That is, catheter 1000 may be formed to include a plurality of fiber optic bundles 1098A, 1098B, 1098C, where each fiber optic bundle 1098A, 1098B, 1098C is formed from a plurality of distinct, fiber optic lines 1035. Each fiber optic bundle 1098A, 1098B, 1098C are disbursed within elongated shaft 1005 circumferentially around fluid drainage lumen 1020. Additionally, each fiber optic bundle 1098A, 1098B, 1098C may be circumferentially spaced apart from adjacent, distinct fiber optic bundles 1098A, 1098B, 1098C. In non-limiting examples, each fiber optic bundle 1098 may be formed from the same number of fiber optic lines 1035, or alternatively may be formed from a distinct number of fiber optic lines 1035 in each bundle. For example, and as shown in the embodiments of FIGS. 25-27, first fiber optic bundle 1098A may include or be formed from two distinct fiber optic lines 1035, second fiber optic bundle 10986 may be formed from four distinct fiber optic lines 1035, and third fiber optic bundle 1098C may be formed from three distinct fiber optic lines 1035. Each fiber optic bundle 1098A, 10986, 1098C may extend from first end 1010 of elongated shaft 1005 toward lens 1090. In one example (see, FIG. 25), each fiber optic bundle 1098 may extend between first end 1010 of elongated shaft 1005 and lens 1090 at an equal length or distance. In other embodiments (see, FIG. 27), each fiber optic bundle may extend within elongated shaft 1005 at different/distinct, predetermined lengths from one another. For example, fiber optic bundle 1098B may extend to be directly adjacent lens 1090 (e.g., longest length), while the length of fiber optic bundle 1098A may be smaller than both fiber optic bundles 1098B, 1098C.

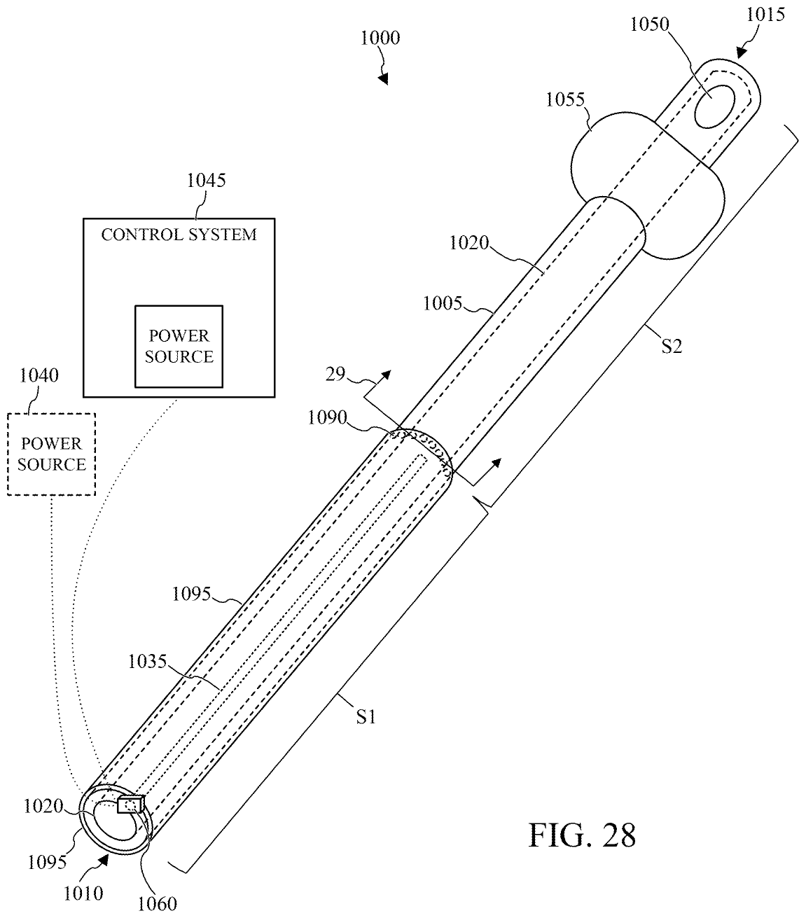

FIGS. 28-32B show further non-limiting examples of catheter 1000. More specifically, FIGS. 28-32B show non-limiting examples of catheter 1000 including a plurality of lenses 1090 formed therein. It is understood that similarly named components and/or similarly numbered components may function in a substantially similar fashion, may include similar materials/components, and/or may include similar interactions with other components. Redundant explanation of these components has been omitted for clarity.

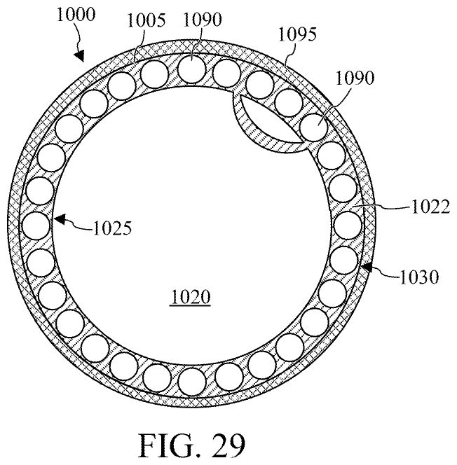

Turning to FIGS. 28 and 29, catheter 1000 may include a plurality of lenses 1090 formed therein. Each of the plurality of lenses 1090 may be positioned within elongated shaft 1005 between first end 1010 and second end 1015. More specifically, each of the plurality of lenses 1090 may be formed, positioned, disposed, and/or included in elongated shaft 1005 between first end 1010 and second end 1015, and may be disposed at least partially/circumferentially around and/or positioned radially adjacent to fluid drainage lumen 1020. In the non-limiting example (see, FIG. 29), each of the plurality of lenses 1090 may also be positioned adjacent one another as well. Each of the Lens 1090 may be formed from any suitable optical lens or transmissive optical device that may disperse, spread, scatter, and/or diffuse the light generated by light source 1060 and provided to catheter 1000 via fiber optic line 1035. In the non-limiting example, and as shown in FIG. 29, the plurality of lenses 1090 may be disposed, formed, and/or included within wall 1022 of elongated shaft 1005. Additionally, each of the plurality of lenses 1090 may be formed, positioned, and/or disposed between inner surface 1025 and outer surface 1030 of elongated shaft 1005 as well.

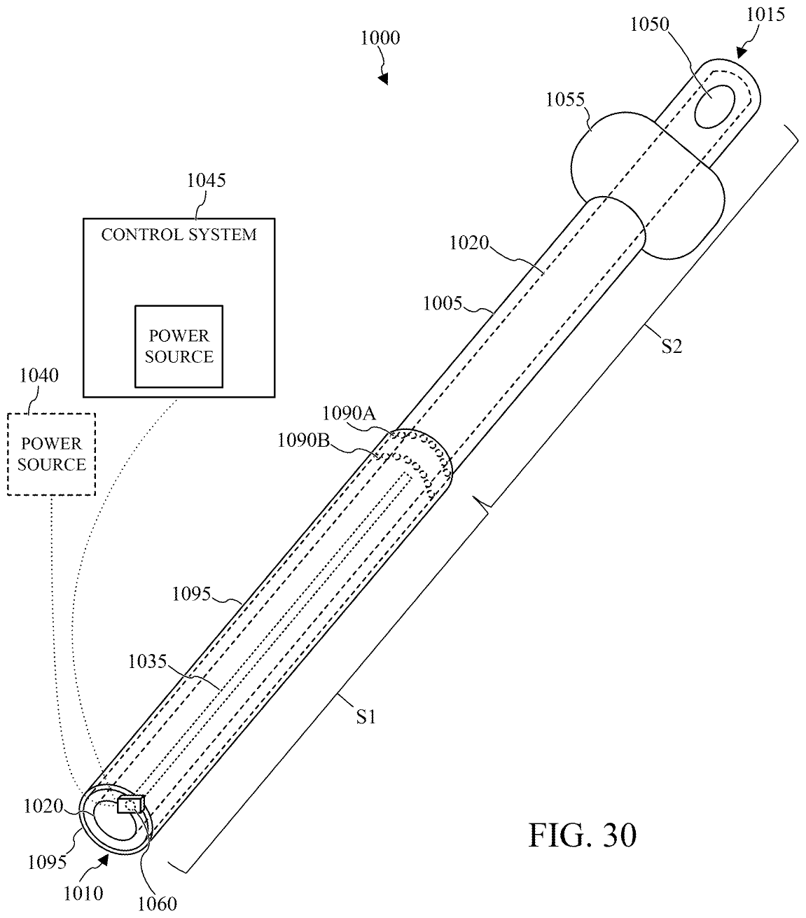

Although a single group or row of lenses 1090 are shown, it is understood that catheter 1000 may include multiple groups or rows of lenses 1090. For example, and as shown in FIG. 30, catheter 1000 may include two groups of a plurality of lenses 1090A, 1090B, where each group of lenses 1090A, 1090B may be positioned within first section (S1) of elongated shaft 1005. Additionally, each group of lenses 1090A, 1090B may be axially spaced apart from one another within elongated shaft 1005. That is, each group of the plurality of lenses 1090A, 1090B positioned within elongated shaft 1005 of catheter 1000 may be axially spaced or separated from one another to aid in the transmission, dissipation, and/or disbursement of light within second section (S2), as discussed herein. The (axial) spacing or distance between each group of lenses 1090A, 1090B within catheter 1000 may be predetermined and/or based on various characteristics including, but not limited to, characteristics (e.g., intensity) of the light generated by light source 1060, characteristics of fiber optic line 1035 (e.g., length, size, number of fiber optic lines), characteristics of lenses 1090A, 1090B (e.g., size, number, position), and/or characteristics of the patient.

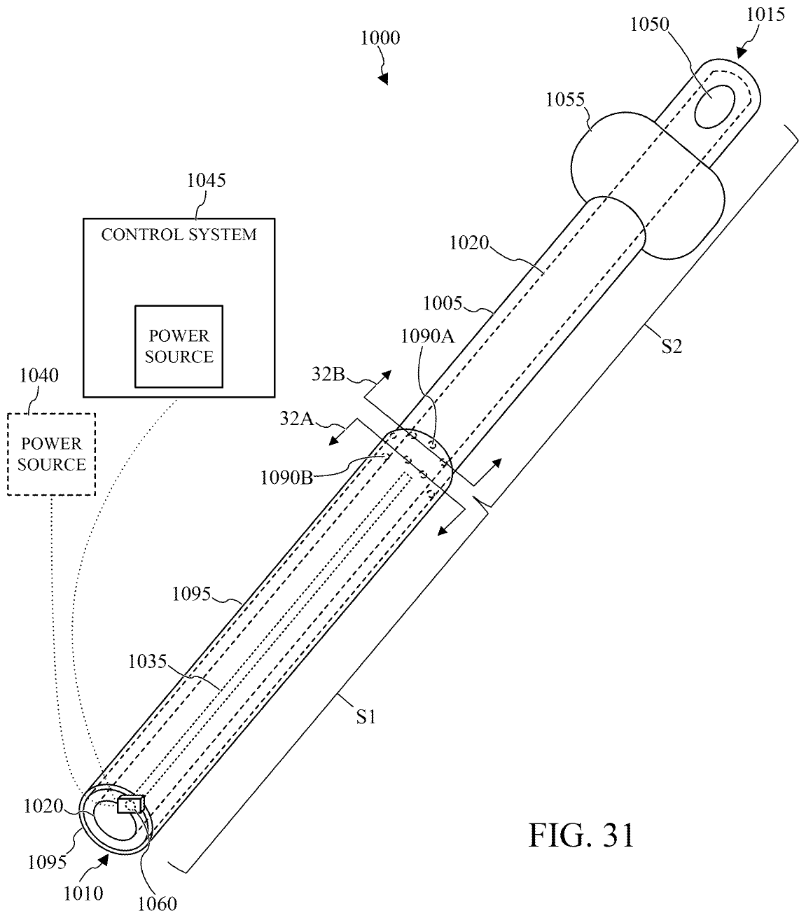

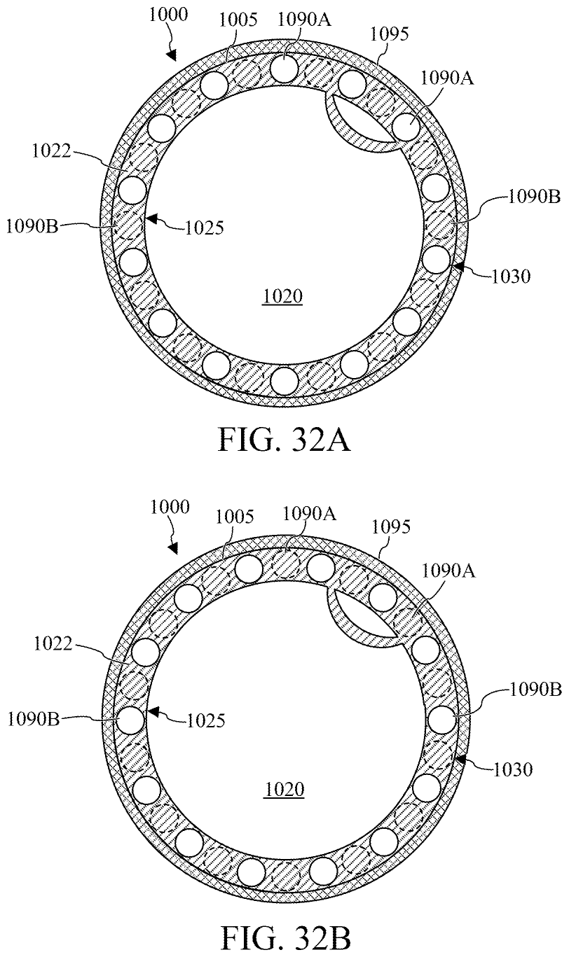

Lenses 1090 may also be axially staggered and/or in an alternating pattern within elongated shaft 1005 of catheter 1000. For example, and with reference to FIGS. 31-32B, catheter 1000 may include two groups of a plurality of lenses 1090A, 1090B, where each group of lenses 1090A, 1090B may be positioned within first section (S1) of elongated shaft 1005, and each group of lenses 1090A, 1090B may be axially spaced apart from one another within elongated shaft 1005. However in the non-limiting example, no two lenses in each group of lenses 1090A, 1090B may be axially aligned. That is, each lens of the first group of lenses 1090A may not be axial aligned and/or may be offset from each lens of the second group of lenses 1090B. As such, light generated by light source 1060 and transmitted through first section (S1) of elongated shaft 1005 via fiber optic line(s) 1035 may pass through first group of lenses 1090A and second group of lenses 1090B before being transmitted to second section (S2), but not necessarily both groups of lenses 1090A, 1090B based on the staggered configuration.

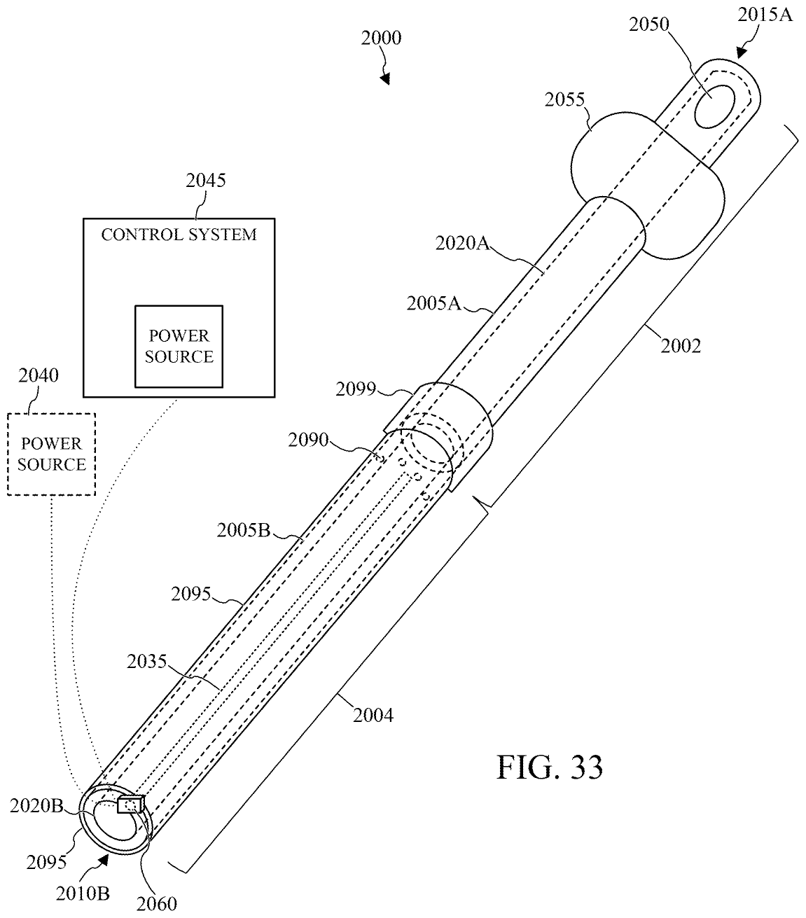

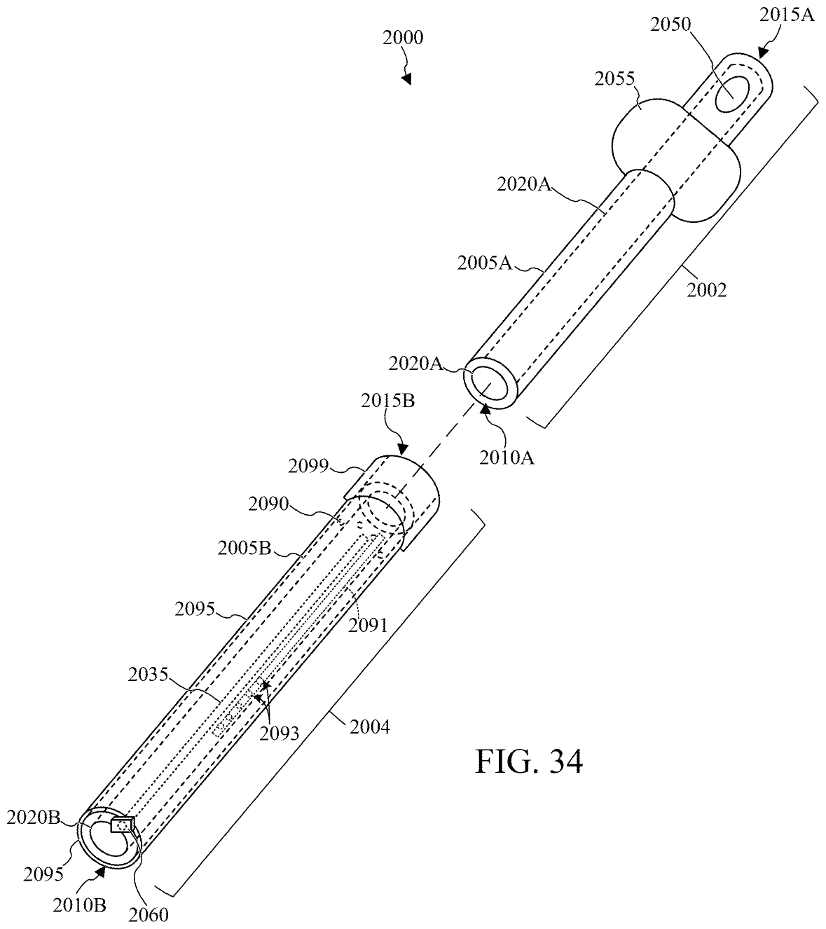

FIGS. 33 and 34 show perspective views of a catheter system 2000. More specifically, FIG. 33 shows a perspective view of an assembled catheter system 2000, while FIG. 34 shows a perspective, exploded view of catheter system 2000. It is understood that similarly named components and/or similarly numbered components may function in a substantially similar fashion, may include similar materials/components, and/or may include similar interactions with other components. Redundant explanation of these components has been omitted for clarity.

In the non-limiting example catheter system 2000 may include a catheter section 2002 and a fiber optics section 2004. As shown, each section 2002, 2004 may be formed as separate/distinct components, where catheter section 2002 may be coupled to fiber optics section 2004 during operation (see, FIG. 33). More specifically, first elongated shaft 2005A of catheter section 2002 may include a first end 2010A and a second end 2015A, while second elongated shaft 2005B of fiber optics section 2004 may include first end 2010B and second end 2015B. First end 2010A of catheter section 2002 may be coupled, affixed, and/or connected to second end 2015B of fiber optics section 2004. In the non-limiting example shown, fiber optics section 2004 may include a slot 2099 for receiving first end 2010A of catheter section 2002, and/or aligning catheter section 2002 and fiber optics section 2004. Additionally in a non-limiting example, slot 2099 of fiber optics section 2004 may also aid in the (releasable) coupling of catheter section 2002 and fiber optics section 2004. Catheter section 2002 and fiber optics section 2004 of catheter system 2000 may be coupled using any suitable coupling components and/or technique including, but not limited to, compression fit, threaded coupling configurations, surgical adhesives, turn-lock mechanism/configuration, and/or the like.

Turning to FIG. 34, and as discussed herein, slot 2099 may also align catheter section 2002 and fiber optics section 2004. More specifically, slot 2099 may align a first fluid drainage lumen 2020A of catheter section 2002 with a second fluid drainage lumen 2020B of fiber optics section 2004, such that the second fluid drainage lumen 2020B of second elongated shaft 2005B is in fluid communication with first fluid drainage lumen 2020A of second elongated shaft 2005A. Additionally, slot 2099 may also align the walls 2022A, 2022B forming each of the first elongated shaft 2005A and second elongated shaft 2005B. Aligning walls 2022A, 2022B allows for light generated from light source 2060 to be dispersed, transmitted, and/or propagated from fiber optics section 2004, and more specifically fiber optic line 2035/lens 2090, through catheter section 2002.

In the non-limiting example shown in FIGS. 33 and 34, catheter section 2002 may correspond to and/or may include similar portions, components, and/or devices as second section (S2) of catheter 1000 discussed herein with respect to FIGS. 17-32B. Additionally, fiber optics section 2004 may correspond to and/or may include similar portions, components, and/or devices as first section (S1) of catheter 1000 discussed herein with respect to FIGS. 17-32B. As similarly discussed herein, catheter section 2002 may include the portion of catheter system 2000 that may be inserted and positioned within a patient's urethra, while fiber optics section 2004, including cover 2095, lens 2090, fiber optic line 2035, and light source 2060, may remain outside of the patient's body. As such, catheter section 2002 of catheter system 2000 may include a predetermined length based on characteristics of the patient. Characteristics of the patient may include, but are not limited to, gender, injury, stage of injury, or other prevalent characteristics.

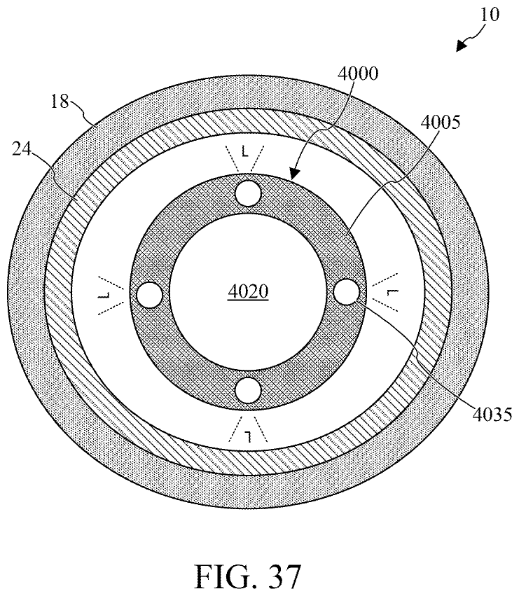

As further shown in the embodiment of FIG. 34, the catheter 2000 can be embodiment with a passage 2091 for a curable material, such as a photocurable gelatin 24 (FIG. 37), can be selectively passed therethrough and emitted from one or more apertures 2093 within a biological fluid passage, such as a ureter 18, artery, vein, or other passage. The passage of the curable material can occur with the light emitting source active such that curing begins the moment the material is emitted from the apertures 2093. Alternatively, the material can be emitted first to be in place within he biological fluid passage and then the light emitter can be activated to starting the curing process.

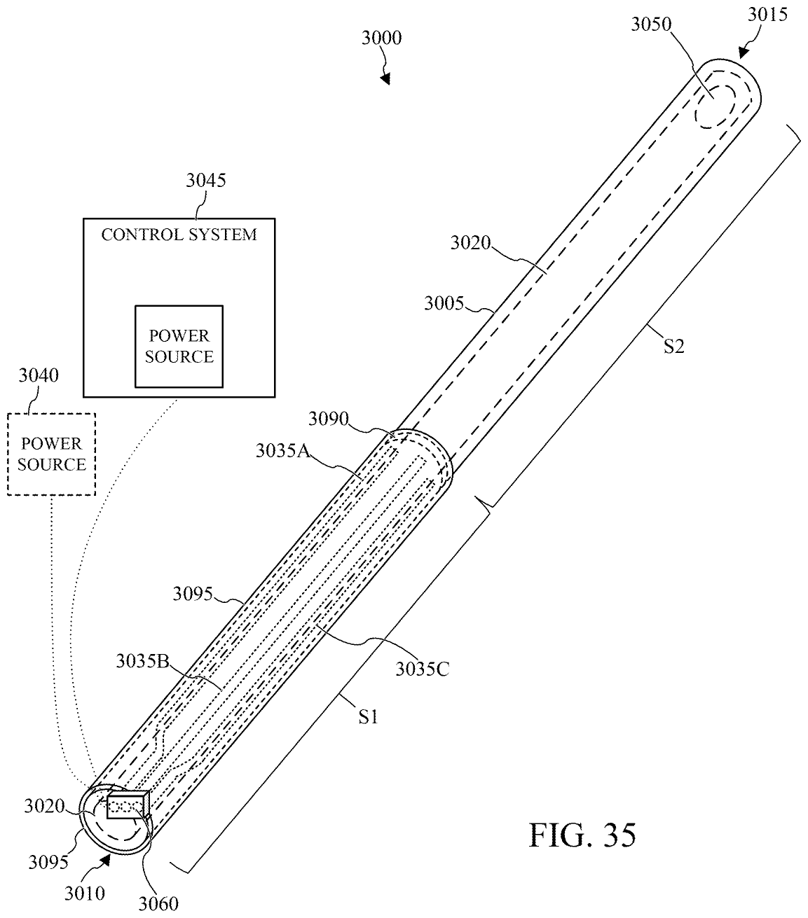

FIG. 35 shows another non-limiting example of catheter device 3000. Specifically, FIG. 35 shows a perspective view of a catheter-type therapy device 3000. Catheter-type therapy device 3000 may be substantially similar to catheter 1000 and/or may include substantially similar features as those discussed herein with respect to catheter 1000 as shown in FIG. 22. It is understood that similarly named components and/or similarly numbered components may function in a substantially similar fashion, may include similar materials/components, and/or may include similar interactions with other components. Redundant explanation of these components has been omitted for clarity.

In the non-limiting example, catheter-type therapy device 3000 (hereafter, “therapy device 3000”) may not include balloon (e.g., balloon 1055), as similarly discussed herein with respect to other catheters (e.g., catheter 1000). Furthermore, and as shown in the non-limiting example of FIG. 35, therapy device 3000 may include fluid drainage lumen 3020 extending through elongated shaft 3005 in phantom as optional. Additionally, catheter or drainage eye 3050 and the opening of fluid drainage lumen 3020 formed adjacent first end 3010 may also be shown in phantom as optional.

That is, therapy device 3000 may or may not include a system (e.g., drainage eye 3050 and fluid drainage lumen 3020) formed in/through elongated shaft 3005 used to remove urine from the bladder through therapy device 3000 while positioned in a patient's urethra. This may be a result of the distinct use and/or implementation of therapy device 3000 when compared to other catheters discussed herein. For example, therapy device 3000 may be inserted into a patient's urethra only to administer light therapy to the urethra. Once the therapy process is complete, therapy device 3000 may be immediately removed from the patient. As such, and because therapy device 3000 may only be required to be positioned within a patient for the time it takes to provide therapy (e.g., 2-20 minutes), fluid drainage lumen 3020 may not be present. In this example, elongated shaft 3005 of therapy device may be a solid, substantially flexible rod or tube that include similar properties and/or characteristics (e.g., light reflective/refractive characteristics) as similarly discussed herein.

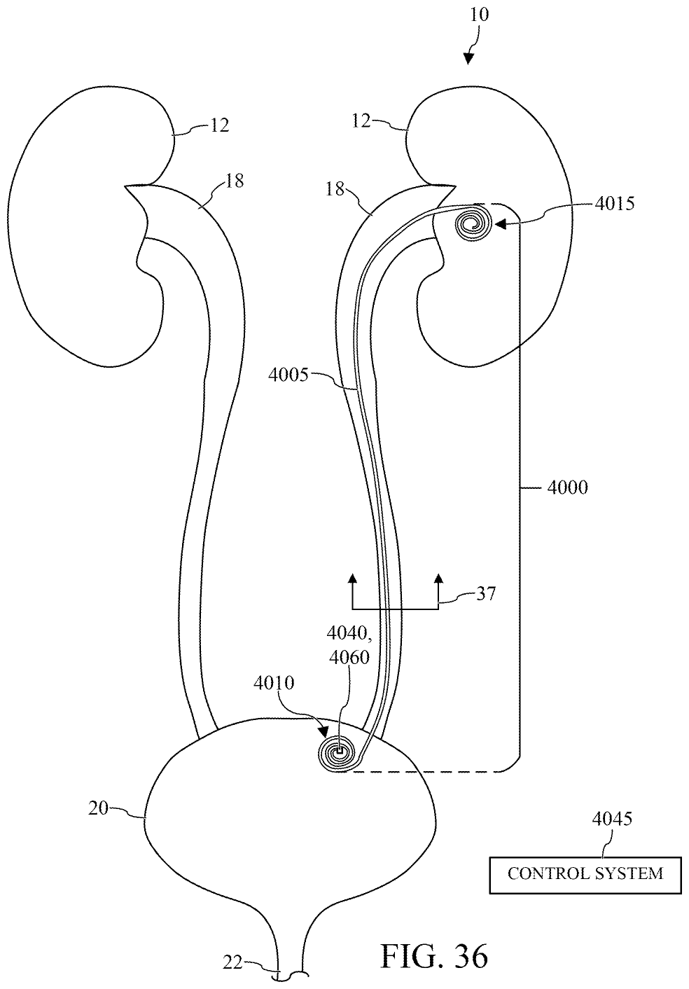

FIG. 36 shows a schematic view of a urinary system 10 for a patient. As shown, urinary system 10 may include two kidneys 12, each kidney 12 in fluid communication with a bladder 20 via distinct ureters 18. Bladder 20 may also include and/or be in fluid communication with the patient's urethra 22.

Additionally, FIG. 36 shows a ureteral stent 4000. Ureteral stent 4000 may include similar features and/or components as those discussed herein with respect to catheter 1000 with reference to FIGS. 17-32. As shown, ureteral stent 4000 may include elongated shaft 4005 having a first end 4010 and a second end 4015 formed opposite first end 4010. In the non-limiting example, first end 4010 of ureteral stent 4000 may be positioned within bladder 20, while second end 4015 may be positioned within one of the patient's kidneys 12. As such, elongated shaft 4005 of ureteral stent 4000 may also extend between kidney 12 and bladder 20, within ureter 18 connecting kidney 12 and bladder 20. In the example, first end 4010 and second end 4015 may include, be formed, and/or configured as at least one curl or turn within elongated shaft 4005. Curl(s) or turn(s) may aid in the retention of elongated shaft 4005 of ureteral stent 4000 when implanted within the patient's urinary system 10.

Ureteral stent 4000 may also include a power source 4040 and light source 4060. In the non-limiting example shown in FIG. 36, power source 4040 and light source 4060 may be positioned on and/or adjacent first end 4010 of elongated shaft 4005. As shown, power source 4040 and light source 4060 may be positioned outside of elongated shaft 4005, and/or may be positioned within bladder 20 when ureteral stent 4000 is implanted into a patient's urinary system 10. Briefly turning to FIG. 37, ureteral stent 4000 may also include a plurality of fiber optic lines 4035 formed within elongated shaft 4005, adjacent fluid drainage lumen 4020. In the non-limiting example, fiber optic line 4035 may extend through at least a portion of elongated shaft 4005. For example, fiber optic line 4035 may extend completely through elongated shaft 4005, from first end 4010 to second end 4015, or alternatively, may extend from first end 4010 toward second end 4015, but ending proximate or a predetermined distance from second 4015. As similarly discussed herein, light source 4060, which may be powered/operational by power source 4040, may be in electrical and/or optical communication with each fiber optic line 4035. Light source 4060 may provide a light through each fiber optic line 4035, and in turn through elongated shaft 4005 of ureteral stent 4000 to provide light therapy to a patient. Although shown and described herein as combined features and/or components, it is understood that power source 4040 and light source 4060 may be formed as distinct components or features.

A control system 4045, external to ureteral stent 4000, may be in communication with ureteral stent 4000 to control the operation during the light therapy procedure. For example, control system 4045 may be in electronic communication with power source 4040 and/or light source 4060 of ureteral stent 4000. After implantation of ureteral stent 4000 within urinary system 10, control system 4045 may send a signal or communicate with power source 4040 and/or light source 4060 to operate light source 4060 for providing light therapy to the patient.

In other non-limiting examples, ureteral stent 4000 may also include a cover (not shown), formed over a portion of elongated shaft 4005. As similarly discussed herein with respect to FIGS. 17-32, the cover of ureteral stent 4000 may be disposed, enclose, and/or substantially surround a section of elongated shaft 4005. The section of elongated shaft 4005 covered by cover may include fiber optic lines 4035. In a non-limiting example, the section covered by the cover may include a section disposed or positioned within bladder 20, such that cover may end on a portion of elongated shaft 4005 positioned within the patient's ureter 18.

FIG. 37 shows a cross-sectional front view of patient's ureter 18 and elongated shaft 4005 of ureteral stent 4000 taken along line 37. In the non-limiting example, and as discussed herein, elongated shaft 4005 of ureteral stent 4000 may extend through and/or be positioned with ureter 18 to provide light therapy to a patient's urinary system 10. To aid treating and/or preventing the development of scarring, narrowing, and/or strictures in hollow tubular structures (e.g., ureter 18), ureteral stent 4000 may provide light therapy (Light L) in conjunction with additional treatments and/or medical interventions. For example, a photocurable gelatin 24 may be disposed within a patient's ureter 18, prior to or after the implantation of ureteral stent 4000, but before performing the light therapy procedure.

As shown in FIG. 37, photocurable gelatin 24 may substantially coat or be disposed around an inner surface of ureter 18—adjacent to elongated shaft 4005 of ureteral stent 4000. Upon applying a light therapy (Light L) and/or emitting material-curative light within ureter 18 using ureteral stent 4000, photocurable gelatin 24 may further aid in treating and/or preventing the formation of strictures within ureter 18. Support for improved stricture treatment using photocurable gelatin 24, as well as examples of photocurable gelatin 24, may be found in “Ability of photocurable gelatin to prevent stricture recurrence after urethral dilation in rabbits,” K. Ojima et al., International Journal of Urology, 2021, the content of which is hereby incorporated by reference into the present application.

The material-curative light can be in red, near-red, infrared, ultraviolet, or any other energetic light wavelength sufficient to cure a material from a liquid to a solid or semisolid, or otherwise effect a desired phase change within a material. Furthermore, the catheter can include a second passage or other means to allow the selective emission of curable material, such as photocurable gelatin 24, within a biological passage, such as a ureter 18. In such embodiment, one or more curable material apertures can

[symbol]

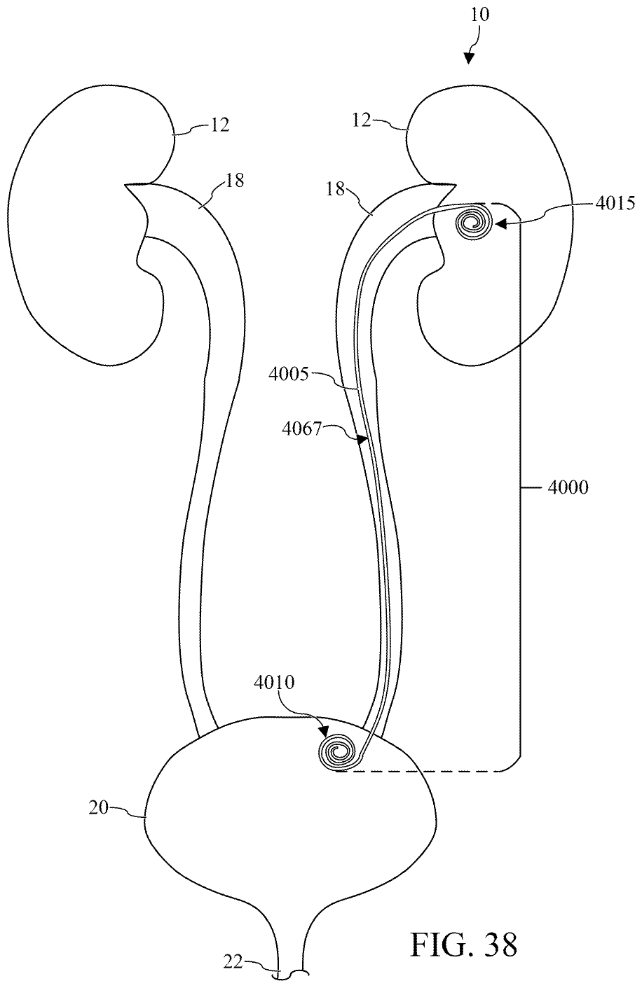

FIG. 38 shows another non-limiting example of urinary system 10 for a patient and ureteral stent 4000. It is understood that similarly named components and/or similarly numbered components may function in a substantially similar fashion, may include similar materials/components, and/or may include similar interactions with other components. Redundant explanation of these components has been omitted for clarity.

In the non-limiting example of ureteral stent 4000 shown in FIG. 38, stent 4000 may provide light therapy via a bio-chemical reaction once implanted within the patient's urinary system 10. That is, and with comparison to the non-limiting example shown in FIG. 36, ureteral stent 4000 may not include power source 4040 and/or light source 4060 for providing illumination/light therapy. Additionally, stent 4000 may also not include fiber optic lines 4035 as well. Rather, elongated shaft 4005 may be substantially filled and/or may include a bio-chemical compound or combination 4067. Bio-chemical combination 4067 may be activated and/or may be mixed within elongated shaft 4005 to create a bio/chemical reaction to produce a light/luminance within elongated shaft 4005 of ureteral stent 4000. The bio/chemical reaction may be achieved prior to implanting ureteral stent 4000, or alternatively may be achieved as elongated shaft 4005 is flexed, bent, and/or positioned within the patient's urinary system 10. The light emitted and/or luminance properties exerted by bio-chemical combination 4067 in elongated shaft 4005 of ureteral stent 4000 shown in FIG. 38 may be substantially similar to the light provided by light source 4060 and fiber optic lines 4035.

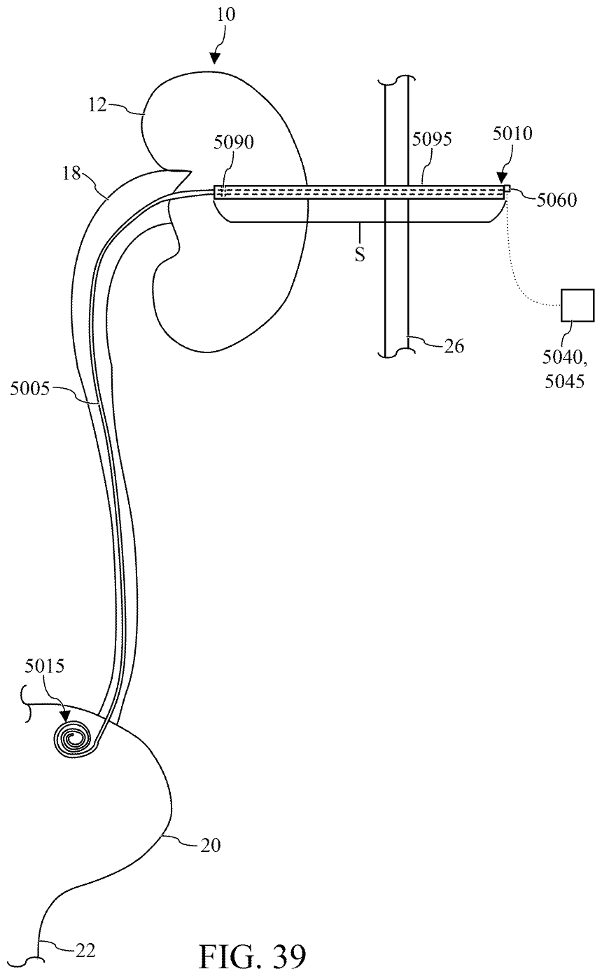

FIG. 39 shows a non-limiting example of urinary system 10 for a patient and a nephroureteral stent 5000. Nephroureteral stent 5000 may include substantially similar features as catheter 1000 shown and discussed herein with respect to FIG. 17 and/or ureteral stent 4000 shown and discussed herein with respect to FIG. 36.

Distinct from the non-limiting example shown and discussed herein with respect to FIG. 36, second end 5015 of nephroureteral stent 5000 may be positioned inside bladder 20. Additionally, first end 5010, formed opposite second end 5015, may be positioned outside of urinary system 10. More specifically, and as shown in FIG. 39, first end 5010 of nephroureteral stent 5000 may be positioned outside of a patient's body or skin 26 — adjacent to kidney 12. Light source 5060 nephroureteral stent 5000 of may be positioned on and/or adjacent first end 5010 of elongated shaft 5005, outside of patient's skin 26. Power source 5040 and control system 5045 may also be positioned outside of patient's body 26. As shown, power source 5040 and control system 5045 may be in electronic communication with light source 5060 to control the operation of light source 5060. Although not shown, elongated shaft 5005 nephroureteral stent 5000 may also include at least one fiber optic line extending from first end 5010, through a section (S) of elongated shaft 5005 toward second end 5015. In the non-limiting example shown in FIG. 39, section (S) of elongated shaft 5005 including at least one fiber optic line may also include a cover 5095. Cover 5095 may be substantially disposed over, enclose, and/or substantially surround a section (S) of elongated shaft 5005 including fiber optic lines. As discussed herein, cover 5095 may direct light generated by the fiber optic lines through elongated shaft 5005 to a desired site on the patient (e.g., stricture) and/or may prevent the light from undesirably dissipating from elongated shaft 5005 before being emitted to the desired site on the patient.

To aid in the directing, spreading, reflecting, and/or refracting, nephroureteral stent 5000 may also include at least one lens 5090. Len(s) 5090 may be formed, positioned, disposed, and/or included in elongated shaft 5005 between first end 5010 and second end 5015, and may be disposed at least partially/circumferentially around and/or positioned adjacent to fluid drainage lumen. As shown, len(s) 5090 may positioned or formed adjacent an end of cover 5095, opposite first end 5010.

Although shown and discussed herein with reference to treating and/or preventing the development of scarring, narrowing, and/or strictures in the urinary system 10 (e.g., urethra 22, ureter 18, bladder 20, kidney 12), it is understood that catheters and therapy devices discussed herein with reference to FIGS. 1-39 may be used in any hollow tubular structures of the body. That is, the catheters and therapy devices discussed herein may be treat or prevent strictures in any hollow tubular structure or hollow organ. For example, the catheters/therapy devices discussed herein may be implemented/used and perform light therapy processes on a patient's esophagus, trachea, stomach, colon, or the like.

The disclosure of a therapeutic light catheter described herein may be wherein the parameters may be adjusted to achieve acceptable characteristics by those skilled in the art by utilizing the teachings disclosed herein. While the foregoing is directed to embodiments of the present disclosure, other and further embodiments of the disclosure may be devised without departing from the basic scope thereof.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. “Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

What is claimed is:

1. A system of providing therapeutic light from a lighted catheter within a biological fluid passage, comprising:
a catheter including an elongated shaft having a first end and a second end positioned opposite the first end, the elongated shaft including a lumen extending substantially between the first end and the second end therein and at least one light emitting element positioned within the elongated shaft, the catheter configured to be placed within a biological fluid passage of a human body; and
a control system in electronic communication with the light emitting element, the control system configured to provide power to the at least one light emitting element such that the powered light emitting element applies a light therapy to a biological fluid passage.

2. The system of claim 1, wherein the light therapy is applied at predetermined time intervals.

3. The system of claim 1, wherein the light emitting element emits at least one of red, near red, or infrared light.

4. The system of claim 1, wherein the light emitting element emits a light at a wavelength between 625 nm and 1 mm.

5. The system of claim 1, wherein the light therapy is applied at a predetermined intensity.

6. The system of claim 1, wherein the light emitting element is a fiber optic line.

7. The system of claim 1, further including a plurality of light emitting elements within the elongated shaft.

8. A system of providing a material-curative light from a lighted catheter within a biological fluid passage, comprising:
a catheter including an elongated shaft having a first end and a second end positioned opposite the first end, the elongated shaft including a lumen extending substantially between the first end and the second end therein, the catheter configured to be placed within a biological fluid passage of a human body;
at least one light emitting element positioned within the elongated shaft the at least one light emitting element selectively emitting material-curative light; and
a control system in electronic communication with the light emitting element, the control system configured to provide power to the at least one light emitting element such that the powered light emitting element selectively emits material-curative light within a biological fluid passage.

9. The system of claim 8, wherein the material-curative light is applied at predetermined time intervals.

10. The system of claim 8, wherein the light emitting element emits at least one of red, near red, infrared, or ultraviolet light.

11. The system of claim 8, with the catheter further including a means of applying a curable material within a biological passage.

12. The system of claim 8, wherein the curative light is applied at a predetermined intensity.

13. The system of claim 8, wherein the light emitting element is a fiber optic line.

14. The system of claim 8, further including a plurality of light emitting elements within the elongated shaft.

15. A method of curing material within a biological fluid passage with a lighted catheter, comprising:
inserting a catheter into a biological fluid passage, the catheter including an elongated shaft having a first end and a second end positioned opposite the first end, the elongated shaft including a lumen extending substantially between the first end and the second end therein
applying a curable material within the biological passage;
curing the curable material with material-curative light emitted from at least one light emitting element positioned within the elongated shaft of the catheter; and
controlling the curing with a control system in electronic communication with the light emitting element, the control system configured to provide power to the at least one light emitting element such that the powered light emitting element selectively emits material-curative light within a biological fluid passage.

16. The method of claim 15, wherein the catheter further including a means of applying a curable material within a biological passage, and applying a curable material is applying the curable material through the means of the catheter.

17. The method of claim 15, wherein curing the curable material is done by applying material-curative light at predetermined time intervals.

18. The method of claim 15, wherein curing the curable material is done by emitting at least one of red, near red, infrared, or ultraviolet light.

19. The method of claim 15, wherein curing the curable material is done by material-curative light emitted from a fiber optic line.

20. The method of claim 15, wherein curing the curable material is done by material-curative light emitted from a plurality of light emitting elements within the elongated shaft of the catheter.
